(19) United States
(12) Patent Application Publication
(10) Pub. No.: US 2024/0164863 A1
(43) Pub. Date: May 23, 2024

(54) SURGICAL ROBOT POSITIONING SYSTEM AND RELATED DEVICES AND METHODS

(71) Applicant: Virtual Incision Corporation, Lincoln, NE (US)

(72) Inventors: Jay Carlson, Lincoln, NE (US); Derek Eilers, Denver, CO (US); Shane Farritor, Lincoln, NE (US); Nathan Wood, Lincoln, NE (US); Parker Durham, Lincoln, NE (US); Lou Cubrich, Lincoln, NE (US); Jonathan Hannaford, Lincoln, NE (US); Mark Chontos, Lincoln, NE (US); Riley Reynolds, Lincoln, NE (US)

(21) Appl. No.: 18/516,281

(22) Filed: Nov. 21, 2023

Related U.S. Application Data
(60) Provisional application No. 63/384,464, filed on Nov. 21, 2022.

(51) Int. Cl. A61B 90/11; A61B 34/30

(52) U.S. Cl. CPC A61B 90/11; A61B 34/30; A61B 2034/302

(57) ABSTRACT

Gross positioning systems for use in positioning robotic surgical devices, wherein each such system can have a positioning ring, an arm base operably coupled to the positioning ring, an arm assembly operably coupled to the arm base, and a device clamp operably coupled to the arm assembly. Other embodiments include a support arm having an adjustment device coupling a horizontal rod to a vertical rod and a rail attachment device coupling the vertical rod to a surgical table.

### CROSS-REFERENCE TO RELATED APPLICATION(S)

This application claims the benefit under 35 U.S.C. § 119(e) to U.S. Provisional Application 63/384,464, filed Nov. 21, 2022 and entitled “Surgical Robot Positioning System and Related Devices and Methods,” which is hereby incorporated herein by reference in its entirety.

### FIELD

The various embodiments herein relate to robotic surgical systems, and more specifically to surgical robot positioning systems and devices that aid in the gross positioning of surgical devices during surgical procedures. The combination of a gross positioning system with an in vivo surgical device results in an increase in the degrees of freedom of the in vivo device without requiring a significant increase in the size of the device.

### BACKGROUND

The known positioning systems currently used for robotic surgery are large and cumbersome. For example, the Da Vinci SP Surgical System™ takes up a significant portion of the operating room and creates a crowded space over the surgical site, and the system created by Waseda University has bulky motor housings that create a larger than necessary profile. In a further example, the Raven™ mimics current laparoscopic techniques by inserting a single tool (in contrast to the in vivo robot systems used in the other two systems discussed above).

Certain of these known systems include a known, generic spherical mechanism that can be used to reach the extents of the abdominal cavity of a patient. A “spherical mechanism” is a physical mechanism or software application that can cause all end effector motions to pass through a single point, thereby allowing a surgical system to use long rigid tools that perform procedures through incisions that serve as single pivot points. As an example, both COBRASurge and the Raven have mechanical spherical mechanisms, while Da Vinci has a software-based spherical mechanism.

There is a need in the art for an improved positioning system.

### BRIEF SUMMARY

Discussed herein are various gross positioning systems for use with robotic surgical devices such as in vivo surgical devices.

In Example 1, a gross positioning system for use with a robotic surgical device comprises a positioning ring, an arm base rotatably coupled to the positioning ring, an arm assembly operably coupled to the arm base, the arm assembly comprising a first link rotatably coupled to the arm base and a second link rotatably coupled to the first link, and a device clamp rotatably coupled to the second link, wherein the device clamp is sized to receive a body of the robotic surgical device therein such that the body is releasably clamped thereto.

Example 2 relates to the gross positioning system according to Example 1, wherein the arm base comprises a base actuator rotatably coupled to the positioning ring.

Example 3 relates to the gross positioning system according to Example 1, wherein the first link comprises a first arm actuator disposed within the first link and rotatably coupled to the arm base and the second link comprises a second arm actuator disposed within the second link and rotatably coupled to the first link.

Example 4 relates to the gross positioning system according to Example 1, further comprising a port support arm disposed adjacent to the positioning ring.

Example 5 relates to the gross positioning system according to Example 4, further comprising a port attachment structure rotatably attached to the port support arm, wherein the port attachment structure is removably coupleable to a port.

Example 6 relates to the gross positioning system according to Example 4, wherein the port support arm is coupled to the gross positioning system via a connection rod, wherein the connection rod is disposed at an angle in relation to a plane of the positioning ring, wherein the angle can range from about 0 degrees to about 45 degrees.

Example 7 relates to the gross positioning system according to Example 5, wherein the port attachment structure is rotatable in relation to the port support arm at an angle in relation to the port support arm that ranges from about 0 degrees to about 45 degrees.

Example 8 relates to the gross positioning system according to Example 4, wherein the positioning ring and the port support arm are sized to receive a portion of the robotic surgical device movably positioned therethrough.

Example 9 relates to the gross positioning system according to Example 1, wherein the positioning ring comprises a semicircular ring.

In Example 10, a gross positioning system for use with a robotic surgical device comprises a stationary positioning ring, an arm base moveably coupled to the positioning ring, wherein the arm base comprises a base actuator rotatably coupled to the positioning ring, and an arm assembly operably coupled to the arm base. The arm assembly comprises a first link rotatably coupled to the arm base, the first link comprising a first arm actuator disposed within the first link and rotatably coupled to the arm base, and a second link rotatably coupled to the first link, the second link comprising a second arm actuator disposed within the second link and rotatably coupled to the first link. The system further comprises a device clamp rotatably coupled to the second link, wherein the device clamp is sized to receive a body of the robotic surgical device therein such that the body is releasably clamped thereto, and a port support arm disposed distal and adjacent to the positioning ring, the port support arm comprising a port attachment structure rotatably attached to the port support arm, wherein the port attachment structure is removably coupleable to a port.

Example 11 relates to the gross positioning system according to Example 10, wherein the port support arm is coupled to the gross positioning system via a connection rod, wherein the connection rod is disposed at an angle in relation to a plane of the positioning ring, wherein the angle can range from about 0 degrees to about 45 degrees.

Example 12 relates to the gross positioning system according to Example 10, further comprising a port attachment structure rotatably attached to the port support arm, wherein the port attachment structure is removably coupleable to a port.

Example 13 relates to the gross positioning system according to Example 12, wherein the port attachment structure is rotatable in relation to the port support arm around an axis disposed at an angle in relation to the port support arm that ranges from about 0 degrees to about 45 degrees.

Example 14 relates to the gross positioning system according to Example 10, wherein the positioning ring and the port support arm are sized to receive a portion of the robotic surgical device movably positioned therethrough.

Example 15 relates to the gross positioning system according to Example 10, wherein the positioning ring comprises a semicircular ring.

Example 16 relates to the gross positioning system according to Example 15, wherein the semicircular ring comprises a ring structure that extends around about 180 degrees of a circle or a ring structure that extends around about 270 degrees of a circle.

Example 17 relates to the gross positioning system according to Example 10, wherein the positioning ring comprises a full ring.

In Example 18, a support arm comprises a vertical rod, a horizontal rod, an adjustment device coupling the horizontal rod to the vertical rod, and a rail attachment device operably coupled to the vertical rod. The adjustment device comprises an adjustment device body, a vertical rod lumen defined through the adjustment device body, wherein the vertical rod is moveably disposed within the vertical rod lumen, first and second triggers operably coupled to the adjustment device body on opposing sides of the vertical rod lumen, wherein each of the first and second triggers is operably coupleable with the vertical rod, a horizontal rod lumen defined through the adjustment device body, wherein the horizontal rod is moveably disposed within the horizontal rod lumen, and a rotatable component rotatably coupled to the adjustment device body, wherein a portion of the rotatable component is rotatably positionable in the horizontal rod lumen such that the portion is engageable with the horizontal rod. The rail attachment device comprises a rail attachment device body comprising upper rail attachment prongs extending from a distal end of the rail attachment body, a moveable clamp body movably attached to the rail attachment device body, the moveable clamp body comprising lower rail attachment prongs extending from a distal end of the moveable clamp body, a clamping screw threadably coupled to the rail attachment device body and the moveable clamp body, a table engagement body slidably coupled to the rail attachment device body, and a vertical rod lumen defined by the rail attachment device body and the table engagement body, wherein the vertical rod is moveably disposed within the vertical rod lumen, wherein rotation of the clamping screw in one direction causes the moveable clamp body to move into a clamped position and urges the vertical rod into the table engagement body such that the table engagement body is moved into a table engagement position.

Example 19 relates to the support arm according to Example 18, wherein the moveable clamp body is rotatably attached to the rail attachment device body, wherein the moveable clamp body is rotatable between an unclamped position and the clamped position.

Example 20 relates to the support arm according to Example 18, wherein the moveable clamp body is in the clamped position, the upper rail attachment prongs are configured to be disposed in contact with an upper edge of a surgical table rail and the lower rail attachment prongs are configured to be disposed in contact with a lower edge of the surgical table rail.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1A is a perspective view of a robotic surgical system.

FIG. 1B is a perspective view of a robotic surgical device.

FIG. 2A is a perspective view of a robotic surgical device positioning system, including a passive support arm and a gross positioning device with a robotic surgical device attached thereto, according to one embodiment.

FIG. 2B is a perspective view of a passive support arm coupled to an incision port as part of the system of FIG. 2A, according to one embodiment.

FIG. 3A is a perspective view of a robotic surgical device positioning system with a robotic device attached thereto, according to another embodiment.

FIG. 3B is a side view of the robotic surgical device positioning system of FIG. 3A, according to one embodiment.

FIG. 3C is a top view of the robotic surgical device positioning system of FIG. 3A, according to one embodiment.

FIG. 3D is another perspective view of the robotic surgical device positioning system of FIG. 3A, according to one embodiment.

FIG. 4A is a perspective view of a gross positioning device, according to another embodiment.

FIG. 4B is a side view of the gross positioning device of FIG. 4A, according to one embodiment.

FIG. 4C is another side view of the gross positioning device of FIG. 4A, according to one embodiment.

FIG. 5A is a perspective view of a positioning ring assembly, according to one embodiment.

FIG. 5B is a cross-sectional side view of the positioning ring assembly of FIG. 5A, according to one embodiment.

FIG. 6 is a side view of a positioning ring assembly with an arm base (or “actuation assembly”) and positioning ring assembly, according to one embodiment.

FIG. 7A is a perspective view of the inner components of an arm base, according to one embodiment.

FIG. 7B is a cross-sectional side view of a shaft of the arm base, according to one embodiment.

FIG. 8 is a perspective view of a portion of a gross positioning device coupled to an incision port, according to one embodiment.

FIG. 9A is a perspective view of a support arm (or “passive support arm”), according to one embodiment.

FIG. 9B is a perspective view of a cord attachment structure associated with the support arm of FIG. 9A, according to one embodiment.

FIG. 9C is a perspective view of a sphere of the cord attachment structure of FIG. 9B, according to one embodiment.

FIG. 10 is a perspective view of a support arm without a cord attachment structure, according to another embodiment.

FIG. 11A is a perspective view of a rail attachment device, according to one embodiment.

FIG. 11B is a perspective view of another rail attachment device, according to a further embodiment.

FIG. 12A is a perspective top view of a port attachment device, according to one embodiment.

FIG. 12B is a perspective bottom view of the port attachment device of FIG. 12A, according to one embodiment.

FIG. 12C is another perspective bottom view of the port attachment device of FIG. 12A in the clamped position, according to one embodiment.

FIG. 13A is a side view of a gross positioning system in use in the vertical minimum plunge position, according to one embodiment.

FIG. 13B is a side view of the gross positioning system of FIG. 13A in the vertical maximum plunge position, according to one embodiment.

FIG. 14A is a side view of a gross positioning system in use in an angled minimum plunge position, according to one embodiment.

FIG. 14B is a side view of the gross positioning system of FIG. 14A in an angled maximum plunge position, according to one embodiment.

FIG. 15A is a perspective view of another robotic surgical device positioning system, including a passive support arm and a gross positioning device with a robotic surgical device attached thereto, according to a further embodiment.

FIG. 15B is a perspective side view of the robotic surgical positioning system of FIG. 15A, according to one embodiment.

FIG. 15C is a perspective bottom view of the robotic surgical positioning system of FIG. 15A, according to one embodiment.

FIG. 15D is a perspective side view of the robotic surgical positioning system of FIG. 15A in an angled position, according to one embodiment.

FIG. 15E is another perspective side view of the robotic surgical positioning system of FIG. 15D, according to one embodiment.

FIG. 15F is a top view of the robotic surgical positioning system of FIG. 15A, according to one embodiment.

FIG. 16A is a perspective side view of a robotic surgical positioning system with a flexible port (and without a robotic surgical device attached thereto), according to one embodiment.

FIG. 16B is another perspective side view of the robotic surgical positioning system of FIG. 16A, according to one embodiment.

FIG. 16C is a perspective bottom view of the robotic surgical positioning system of FIG. 16A, according to one embodiment.

FIG. 17A is a side view of a robotic surgical positioning system with a flexible port and a wound retractor, according to one embodiment.

FIG. 17B is a perspective bottom view of the robotic surgical positioning system of FIG. 17A, according to one embodiment.

FIG. 18A is a perspective side view of a gross positioning device, according to one embodiment.

FIG. 18B is another perspective side view of the gross positioning device of FIG. 18A, according to one embodiment.

FIG. 19A is a perspective side view of a gross positioning device with an extended arm assembly, according to one embodiment.

FIG. 19B is another perspective side view of the gross positioning device of FIG. 19A, according to one embodiment.

FIG. 19C is a side view of a port support structure of the gross positioning device of FIG. 19A, according to one embodiment.

FIG. 19D is a perspective view of the port support structure of FIG. 19C with a port attachment structure attached thereto, according to one embodiment.

FIG. 20A is a perspective view of a ring attachment component, according to one embodiment.

FIG. 20B is a perspective expanded view of a portion of the ring attachment component of FIG. 20A, according to one embodiment.

FIG. 20C is another perspective expanded view of the portion of the ring attachment component of FIG. 20B, according to one embodiment.

FIG. 20D is a cross-sectional perspective view of the ring attachment component of FIG. 20A, according to one embodiment.

FIG. 20E is a further cross-sectional perspective view of the ring attachment component of FIG. 20A in the open position, according to one embodiment.

FIG. 20F is a further cross-sectional perspective view of the ring attachment component of FIG. 20A in the clamped position, according to one embodiment.

FIG. 21A is a perspective view of a gross positioning device with a semicircular positioning ring, according to one embodiment.

FIG. 21B is a perspective view of a gross positioning device with another semicircular positioning ring, according to a further embodiment.

FIG. 21C is a perspective view of a gross positioning device with a full circle positioning ring, according to one embodiment.

FIG. 22 is a perspective view of a gross positioning system in an angled position, according to one embodiment.

FIG. 23A is an expanded view of an arm assembly, according to one embodiment.

FIG. 23B is another expanded view of the arm assembly of FIG. 23A, according to one embodiment.

FIG. 24A is a side view of a first arm link, according to one embodiment.

FIG. 24B is another side view of the first arm link of FIG. 24A, according to one embodiment.

FIG. 24C is an expanded view of a portion of the first arm link of FIG. 24A, according to one embodiment.

FIG. 24D is an expanded perspective view of the portion of the first arm link of FIG. 24C, according to one embodiment.

FIG. 25 is a side view of a second arm link, according to one embodiment.

FIG. 26A is a perspective view of an arm base (or “actuation assembly”) coupled to a positioning ring, according to one embodiment.

FIG. 26B is a perspective view of the coupling of the arm base of FIG. 26A to the positioning ring, according to one embodiment.

FIG. 26C is a cross-sectional side view of the coupling of the arm base of FIG. 26A to the positioning ring, according to one embodiment.

FIG. 27 is a perspective side view of a gross positioning device with actuators, according to one embodiment.

FIG. 28A is a perspective view of the arm base with an actuator, according to one embodiment.

FIG. 28B is a cross-sectional side view of the actuator of FIG. 28A, according to one embodiment.

FIG. 29A is a perspective side view of an actuator on an arm assembly, according to one embodiment.

FIG. 29B is a cross-sectional side view of the actuator of FIG. 29A, according to one embodiment.

FIG. 30 is a perspective view of a gross positioning device with a device clamp, according to one embodiment.

FIG. 31 is a perspective view of a passive joint of the device clamp, according to one embodiment.

FIG. 32 is a perspective view of a support arm (or “passive support arm”), according to one embodiment.

FIG. 33A is a perspective view of a height adjustment mechanism (or “device”) for a support arm, according to one embodiment.

FIG. 33B is a side view of the height adjustment mechanism of FIG. 33A, according to one embodiment.

FIG. 33C is another side view of the height adjustment mechanism of FIG. 33A, according to one embodiment.

FIG. 33D is another side view of a portion of the height adjustment mechanism of FIG. 33A, according to one embodiment.

FIG. 33E is another side view of the portion of the height adjustment mechanism of FIG. 33D, according to one embodiment.

FIG. 34A is a perspective view of a rail attachment mechanism (or “device”) for a support arm, according to one embodiment.

FIG. 34B is another perspective view of the rail attachment mechanism of FIG. 34A, according to one embodiment.

FIG. 34C is a perspective side view of the rail attachment mechanism of FIG. 34A attached to a surgical table, according to one embodiment.

FIG. 34D is cross-sectional side view of the rail attachment mechanism of FIG. 34A attached to a surgical table, according to one embodiment.

FIG. 34E is another cross-sectional side view of the rail attachment mechanism of FIG. 34A attached to a surgical table, according to one embodiment.

### DETAILED DESCRIPTION

The various embodiments disclosed or contemplated herein relate to a surgical robot positioning system that includes a passive support arm and a gross positioning robotic device. A dexterous in vivo surgical robotic device is coupleable to the gross positioning robotic device such that the positioning system can be used for global orientation of the surgical robotic device within the cavity of a patient as described in further detail herein.

The various gross positioning system implementations disclosed or contemplated herein can be used to automatically grossly position a surgical device inside a cavity of a patient. “Gross positioning,” as used herein, is intended to mean general positioning of an entire moveable surgical device (in contrast to precise movement and placement of the specific components of such a device, such as an arm or end effector). In known robotic surgical systems, the gross positioning of those devices during a surgical procedure can be a challenging task. Further, minimally invasive surgical procedures (using either robotic or non-robotic systems) frequently require a surgical technician to reposition the surgical equipment, such as a laparoscope. Such manual gross repositioning takes time and additional effort. In some cases, the surgical technician is a junior medical student who is not fully trained in laparoscopy. As a result, the repositioning instructions from the surgeon often result in an obstructed and/or fogged view of the surgical site, requiring additional cognitive resources from the surgeon. For example, the Da Vinci@ system as well as known single incision surgical devices often require timely manual repositioning of the patient, the robotic system, or both while performing complicated procedures.

The various gross positioning system embodiments contemplated herein aid in the gross repositioning of surgical devices throughout the procedure without additional intervention or manual repositioning from the surgical staff. The surgical devices may include, for example, any surgical devices that have a device body, rod, or tube configured to be positioned through an incision and at least one robotic arm coupled to or positioned through the device body or tube that is positioned entirely within the cavity of the patient. The gross positioning system embodiments can control the degrees of freedom, azimuth and elevation angle, and roll and translation about the axis of insertion of laparoscopic surgical tools, including robotic laparoscopic surgical tools. As a result, the gross positioning system embodiments disclosed and contemplated herein can grossly position a surgical device through an incision, port, or orifice (including a natural orifice) into a patient cavity, such as the abdominal cavity, with high manipulability, reducing the operative time and stress induced upon the surgical staff. The combination of the external gross positioning system with the internal surgical device system will allow the degrees of freedom of the internal system to effectively increase without increasing the size of the surgical robot/device.

In one implementation, the various systems and devices described and contemplated herein can be used with any single site surgical device or system with an available external positioning fixture, such as a protruding body, rod, tube, or magnetic handle. Further, it is understood that the various embodiments of positioning systems disclosed herein can be used with any other known medical devices, systems, and methods that are positioned through an incision, port, or orifice (including a natural orifice). For example, the various embodiments disclosed herein may be used with any of the medical devices and systems disclosed in U.S. Pat. No. 8,968,332 (issued on Mar. 3, 2015 and entitled “Magnetically Coupleable Robotic Devices and Related Methods”), U.S. Pat. No. 8,834,488 (issued on Sep. 16, 2014 and entitled “Magnetically Coupleable Surgical Robotic Devices and Related Methods”), U.S. Pat. No. 10,307,199 (issued on Jun. 4, 2019 and entitled “Robotic Surgical Devices and Related Methods”), U.S. Pat. No. 9,579,088 (issued on Feb. 28, 2017 and entitled “Methods, Systems, and Devices for Surgical Visualization and Device Manipulation”), U.S. Patent Application 61/030,588 (filed on Feb. 22, 2008), U.S. Pat. No. 8,343,171 (issued on Jan. 1, 2013 and entitled “Methods and Systems of Actuation in Robotic Devices”), U.S. Pat. No. 8,828,024 (issued on Sep. 9, 2014 and entitled “Methods and Systems of Actuation in Robotic Devices”), U.S. Pat. No. 9,956,043 (issued on May 1, 2018 and entitled “Methods and Systems of Actuation in Robotic Devices”), U.S. patent application Ser. No. 15/966,606 (filed on Apr. 30, 2018 and entitled “Methods, Systems, and Devices for Surgical Access and Procedures”), U.S. patent application Ser. No. 12/192,663 (filed on Aug. 15, 2008 and entitled “Medical Inflation, Attachment, and Delivery Devices and Related Methods”), U.S. patent application Ser. No. 15/018,530 (filed on Feb. 8, 2016 and entitled “Medical Inflation, Attachment, and Delivery Devices and Related Methods”), U.S. Pat. No. 8,974,440 (issued on Mar. 10, 2015 and entitled “Modular and Cooperative Medical Devices and Related Systems and Methods”), U.S. Pat. No. 8,679,096 (issued on Mar. 25, 2014 and entitled “Multifunctional Operational Component for Robotic Devices”), U.S. Pat. No. 9,179,981 (issued on Nov. 10, 2015 and entitled “Multifunctional Operational Component for Robotic Devices”), U.S. Pat. No. 9,883,911 (issued on Feb. 6, 2018 and entitled “Multifunctional Operational Component for Robotic Devices”), U.S. patent application Ser. No. 15/888,723 (filed on Feb. 5, 2018 and entitled “Multifunctional Operational Component for Robotic Devices”), U.S. Pat. No. 8,894,633 (issued on Nov. 25, 2014 and entitled “Modular and Cooperative Medical Devices and Related Systems and Methods”), U.S. Pat. No. 8,968,267 (issued on Mar. 3, 2015 and entitled “Methods and Systems for Handling or Delivering Materials for Natural Orifice Surgery”), U.S. Pat. No. 9,060,781 (issued on Jun. 23, 2015 and entitled “Methods, Systems, and Devices Relating to Surgical End Effectors”), U.S. Pat. No. 9,757,187 (issued on Sep. 12, 2017 and entitled “Methods, Systems, and Devices Relating to Surgical End Effectors”), U.S. Pat. No. 10,350,000 (issued on Jul. 16, 2019 and entitled “Methods, systems, and devices relating to surgical end effectors”), U.S. patent application Ser. No. 16/512,510 (filed on Jul. 16, 2019 and entitled “Methods, Systems, and Devices Relating to Surgical End Effectors”), U.S. Pat. No. 9,089,353 (issued on Jul. 28, 2015 and entitled “Robotic Surgical Devices, Systems, and Related Methods”), U.S. Pat. No. 10,111,711 (issued on Oct. 30, 2018 and entitled “Robotic Surgical Devices, Systems, and Related Methods”), U.S. patent application Ser. No. 16/123,619 (filed on Sep. 6, 2018 and entitled “Robotic Surgical Devices, Systems and Related Methods”), U.S. Pat. No. 9,770,305 (issued on Sep. 26, 2017 and entitled “Robotic Surgical Devices, Systems, and Related Methods”), U.S. patent application Ser. No. 15/661,147 (filed on Jul. 27, 2017 and entitled “Robotic Devices with On Board Control & Related Systems & Devices”), U.S. patent application Ser. No. 13/833,605 (filed on Mar. 15, 2013 and entitled “Robotic Surgical Devices, Systems, and Related Methods”), U.S. patent application Ser. No. 13/738,706 (filed on Jan. 10, 2013 and entitled “Methods, Systems, and Devices for Surgical Access and Insertion”), U.S. patent application Ser. No. 14/661,465 (filed on Mar. 18, 2015 and entitled “Methods, Systems, and Devices for Surgical Access and Insertion”), U.S. patent application Ser. No. 15/890,860 (filed on Feb. 7, 2018 and entitled “Methods, Systems, and Devices for Surgical Access and Insertion”), U.S. Pat. No. 9,498,292 (issued on Nov. 22, 2016 and entitled “Single Site Robotic Devices and Related Systems and Methods”), U.S. Pat. No. 10,219,870 (issued on Mar. 5, 2019 and entitled “Single site robotic device and related systems and methods”), U.S. patent application Ser. No. 16/293,135 (filed Mar. 3, 2019 and entitled “Single Site Robotic Device and Related Systems and Methods”), U.S. Pat. No. 9,010,214 (issued on Apr. 21, 2015 and entitled “Local Control Robotic Surgical Devices and Related Methods”), U.S. Pat. No. 10,470,828 (issued on Nov. 12, 2019 and entitled “Local Control Robotic Surgical Devices and Related Methods”), U.S. patent application Ser. No. 16/596,034 (filed on Oct. 8, 2019 and entitled “Local Control Robotic Surgical Devices and Related Methods”), U.S. Pat. No. 9,743,987 (issued on Aug. 29, 2017 and entitled “Methods, Systems, and Devices Relating to Robotic Surgical Devices, End Effectors, and Controllers”), U.S. patent application Ser. No. 15/687,787 (filed on Aug. 28, 2017 and entitled “Methods, Systems, and Devices Relating to Robotic Surgical Devices, End Effectors, and Controllers”), U.S. Pat. No. 9,888,966 (issued on Feb. 13, 2018 and entitled “Methods, Systems, and Devices Relating to Force Control Surgical Systems”), U.S. patent application Ser. No. 15/894,489 (filed on Feb. 12, 2018 and entitled “Methods, Systems, and Devices Relating to Force Control Surgical Systems”), U.S. patent application Ser. No. 14/212,686 (filed on Mar. 14, 2014 and entitled “Robotic Surgical Devices, Systems, and Related Methods”), U.S. patent application Ser. No. 14/334,383 (filed on Jul. 17, 2014 and entitled “Robotic Surgical Devices, Systems, and Related Methods”), U.S. patent application Ser. No. 14/853,477 (filed on Sep. 14, 2015 and entitled “Quick-Release End Effectors and Related Systems and Methods”), U.S. patent application Ser. No. 16/504,793 (filed on Jul. 8, 2019 and entitled “Quick-Release End Effectors and Related Systems and Methods”), U.S. Pat. No. 10,376,322 (issued on Aug. 13, 2019 and entitled “Robotic Device with Compact Joint Design and Related Systems and Methods”), U.S. patent application Ser. No. 16/538,902 (filed on Aug. 13, 2019 and entitled “Robotic Device with Compact Joint Design and Related Systems and Methods”), U.S. patent application Ser. No. 15/227,813 (filed on Aug. 3, 2016 and entitled “Robotic Surgical Devices, System and Related Methods”) U.S. patent application Ser. No. 15/599,231 (filed on May 18, 2017 and entitled “Robotic Surgical Devices, Systems, and Related Methods”), U.S. patent application Ser. No. 15/687,113 (filed on Aug. 25, 2017 and entitled “Quick-Release End Effector Tool Interface”), U.S. patent application Ser. No. 15/691,087 (filed on Aug. 30, 2017 and entitled “Robotic Device with Compact Joint Design and an Additional Degree of Freedom and Related Systems and Methods”), U.S. patent application Ser. No. 15/826,166 (filed on Nov. 29, 2017 and entitled “User controller with user presence detection and related systems and methods”), U.S. patent application Ser. No. 15/842,230 (filed on Dec. 14, 2017 and entitled “Releasable Attachment Device for Coupling to Medical Devices and Related Systems and Methods”), U.S. patent application Ser. No. 16/144,807 (filed on Sep. 27, 2018 and entitled “Robotic Surgical Devices with Tracking Camera Technology and Related Systems and Methods”), U.S. patent application Ser. No. 16/241,263 (filed on Jan. 7, 2019 and entitled “Single-Manipulator Robotic Device With Compact Joint Design and Related Systems and Methods”), U.S. patent application Ser. No. 16/736,329 (filed on Jan. 7, 2020 and entitled “Robotically Assisted Surgical System and Related Devices and Methods”), U.S. patent application Ser. No. 17/368,255 (filed on Jul. 6, 2021 and entitled “Surgical Robot Positioning System and Related Devices and Methods”), U.S. patent Ser. No. 18/064,784 (filed on Dec. 12, 2022 and entitled “Robotic Arm with Hybrid Actuation Assemblies and Related Devices, Systems, and Methods”), U.S. patent application Ser. No. 18/299,129 (filed on Apr. 12, 2023 and entitled “Energy Coupling Mitigation Device and Related Systems and Methods”), U.S. Provisional Application 63/384,496, filed on Nov. 21, 2022, U.S. Provisional Application 63/384,502, filed on Nov. 21, 2022, U.S. Pat. No. 7,492,116 (filed on Oct. 31, 2007 and entitled “Robot for Surgical Applications”), U.S. Pat. No. 7,772,796 (filed on Apr. 3, 2007 and entitled “Robot for Surgical Applications”), and U.S. Pat. No. 8,179,073 (issued on May 15, 2011, and entitled “Robotic Devices with Agent Delivery Components and Related Methods”), all of which are hereby incorporated herein by reference in their entireties.

Certain device and system implementations disclosed in the applications listed above can be positioned within a body cavity of a patient, or a portion of the device can be placed within the body cavity, in combination with a positioning system such as any of the embodiments disclosed or contemplated herein. An “in vivo device” as used herein means any device that can be positioned, operated, or controlled at least in part by a user while being positioned within a body cavity of a patient, including any device that is coupled to a support component such as a rod, tube, body, or other such component that is disposed through an opening or orifice of the body cavity, also including any device positioned substantially against or adjacent to a wall of a body cavity of a patient, further including any such device that is internally actuated (having no external source of motive force), and additionally including any device that may be used laparoscopically or endoscopically during a surgical procedure. As used herein, the terms “robot,” and “robotic device” shall refer to any device that can perform a task either automatically or in response to a command.

In certain implementations, any robotic device that is coupleable to the various positioning system embodiments disclosed or contemplated herein can be positioned through an insertion port. Further, in certain embodiments as discussed in detail below, the positioning system can be coupled to the insertion port. The insertion port can be a known, commercially-available flexible membrane (referred to herein as a “gelport”) placed transabdominally to seal and protect the abdominal incision. This off-the-shelf component is the same device or substantially the same device that is used in substantially the same way for Hand-Assisted Laparoscopic Surgery (HALS). The only difference is that the arms of the robotic device according to the various embodiments herein are inserted into the abdominal cavity through the insertion port rather than the surgeon's hand. The robotic device body, rod, or tube seals against the insertion port when it is positioned therethrough, thereby maintaining insufflation pressure. The port is single-use and disposable. Alternatively, any known port can be used. In further alternatives, the various devices that can be used in combination with the various positioning system embodiments herein can be inserted through an incision without a port or through a natural orifice.

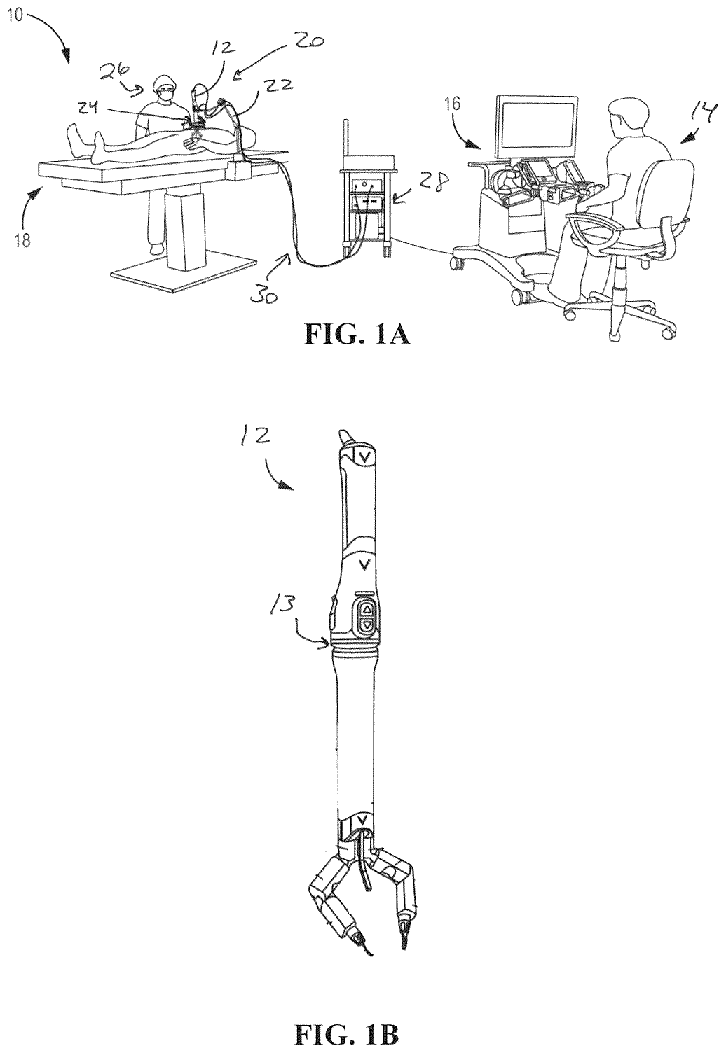

FIG. 1A depicts one embodiment of a robotic surgical system 10 having several components that will be described in additional detail below. The components of the various positioning system implementations disclosed or contemplated herein can be used with a full surgical system 10 that includes an external control console 16 and a robotic surgical device 12. In accordance with the implementation of FIG. 1A, the robotic surgical device 12 is shown mounted to the operating table 18 (or a rail thereof) via a robot positioning system 20 according to one embodiment as described in additional detail below. The robot positioning system 20 has a passive support arm 22 and a robotic positioning device 24 coupled to the arm 22. The support arm 22 is coupled to the operating table 18 and the robotic device 12 is coupleable to the robotic positioning device 24. The system 10 can be, in certain implementations, operated by the surgeon 14 at the console 16 and one surgical assistant 26 positioned at the operating table 18. That is, the surgeon 14 at the console 16 can control both the robotic device 12 and the gross positioning robotic device 24, and the surgical assistant 26 can control the remaining system 10 components (e.g., passive support arm 22). Alternatively, one surgeon 14 can operate the entire system 10. In a further alternative, three or more people can be involved in the operation of the system 10. It is further understood that the surgeon (or user) 14 can be located at a remote location in relation to the operating table 18 such that the surgeon 14 can be in a different city or country or on a different continent from the patient on the operating table 18. The console 16 can be any console as disclosed in any of the various patents and/or applications incorporated by reference above. Alternatively, the console 16 can be any known console for operating a robotic surgical system or device.

In this specific implementation, the robotic device 12 is connected to the interface pod and electrosurgical unit 28 via connection cables 30. Further, the gross positioning robotic device 24 is also coupled to the interface pod and electrosurgical unit 28 via the connection cables 30. Alternatively, any wired or wireless connection configuration can be used. Further, the interface pod and electrosurgical unit 28 is coupled to the console 16 as shown (and alternatively can be coupled via any known wired or wireless connection). In certain implementations, the system 10 can also interact with other devices during use such as auxiliary monitors, etc.

According to various embodiments, the gross positioning robotic device 24 of the positioning system 20 can dock or otherwise couple with the surgical robotic device 12 and control the position of the workspace of the device 12 by supporting and moving the surgical robotic device 12 during a surgical procedure. This allows the surgeon 14 (and the assistant 26) to have complete control of the robotic device 12 with respect to the target surgical area (the target cavity of the patient).

One embodiment of a robotic surgical device 12 is depicted in FIG. 1B. Alternatively, other robotic surgical devices can be used in combination with the various positioning system embodiments disclosed or contemplated herein.

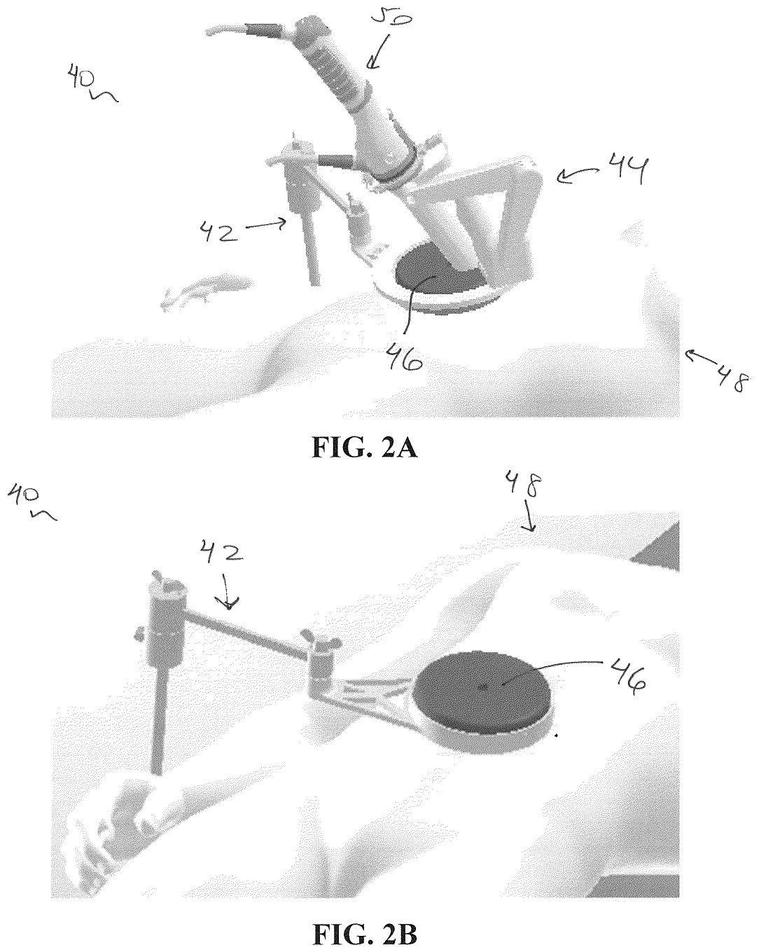

One embodiment of a robotic surgical device positioning system 40 is depicted in FIGS. 2A and 2B. The system 40 includes a passive support arm 42 and a gross robot positioning device 44 coupled to the arm 42. As will described in further detail below, the passive support arm 42 is coupled to an incision port 46 disposed within the incision of the patient 48 (as best shown in FIG. 2B), and the gross robot positioning device 44 is coupled to the support arm 42 at or adjacent to the incision port 46 (as best shown in FIG. 2A). Any known robotic device 50, which in this specific exemplary implementation is represented by the surgical device 50 as shown, can be removably coupled to the positioning device 44 such that the robotic surgical device 50 is disposed through the port 46 into the target cavity of the patient. In certain embodiments, the port 46 is a gelport 46. Alternatively, the port 46 can be any known incision port 46, as will be discuss in further detail below.

The gross positioning device 44 in this implementation—and various other embodiments as disclosed or contemplated herein—is a3 degree-of-freedom (“DOF”) robotic remote center-of-motion (RCM) mechanism, controlling yaw, pitch and plunge of the robotic surgical device 46. It is understood that an RCM is the point about which a rotational joint rotates and further that an RCM mechanism is a device where all the kinematic joints move to adjust the position of the robotic surgical device through the same RCM point. For the various gross positioning devices herein (including the robot positioning device 44), the RCM point is within the workspace of the robot positioning device 44 such that, while the end effector of the robotic surgical device 46 can still desirably be manipulated, there is a point of no relative motion with respect to the rest of the mechanism. More specifically, in many implementations the RCM is approximately located at the incision, port, or orifice during surgical use. For example, the RCM point can is positioned at the port (such as port 46). As such, there is no relative motion at this patient-device interface that could cause harm to the patient, while still allowing the robotic surgical device 50 full access to the target surgical site (a cavity within the patient).

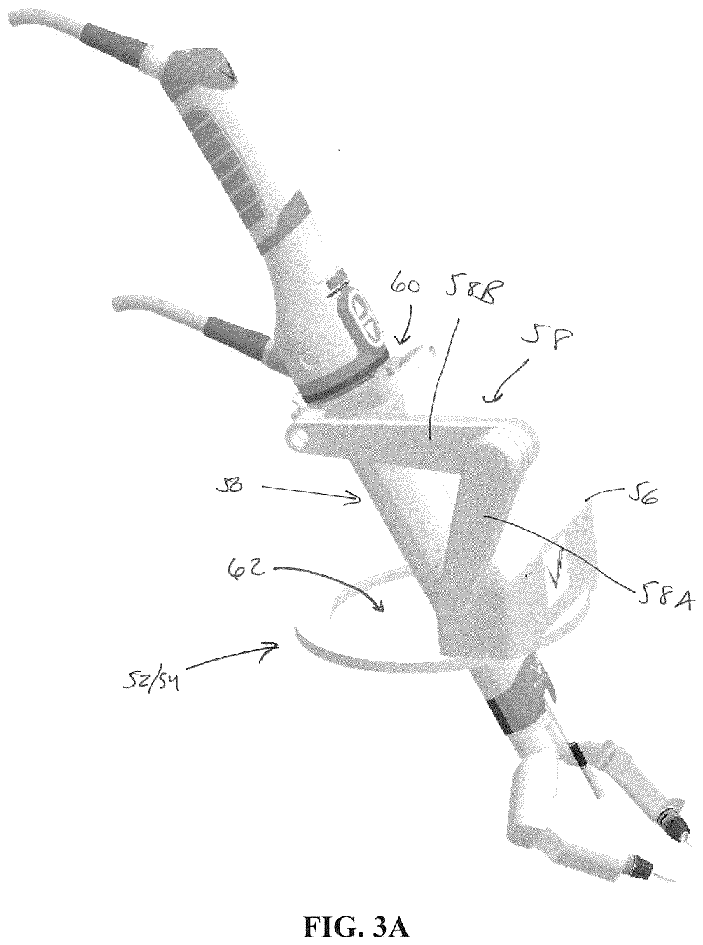

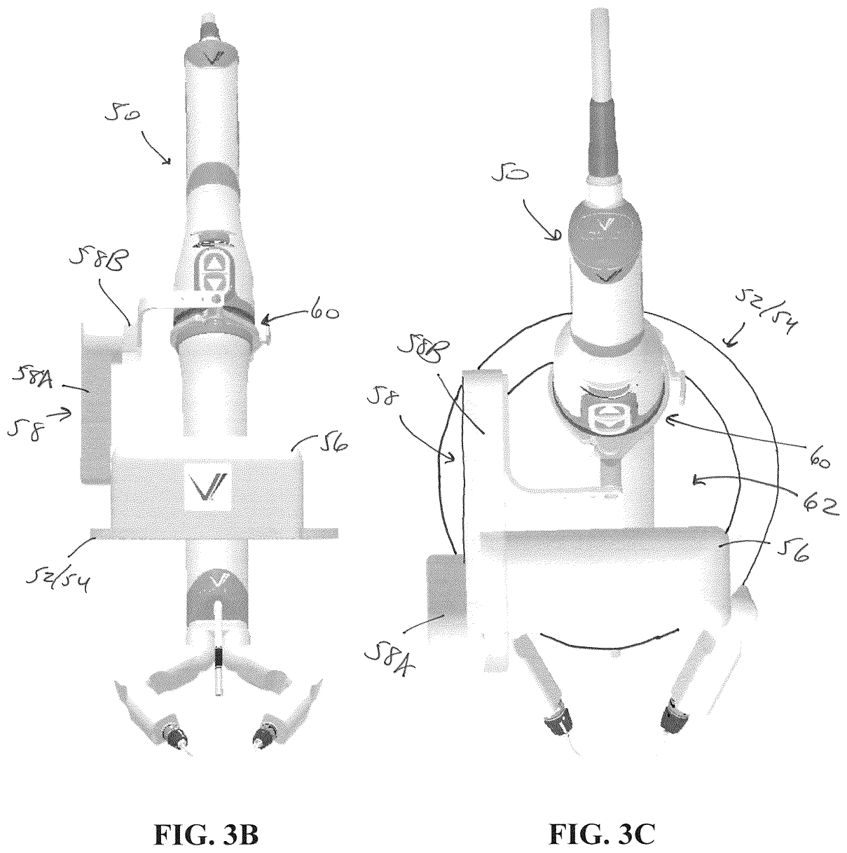

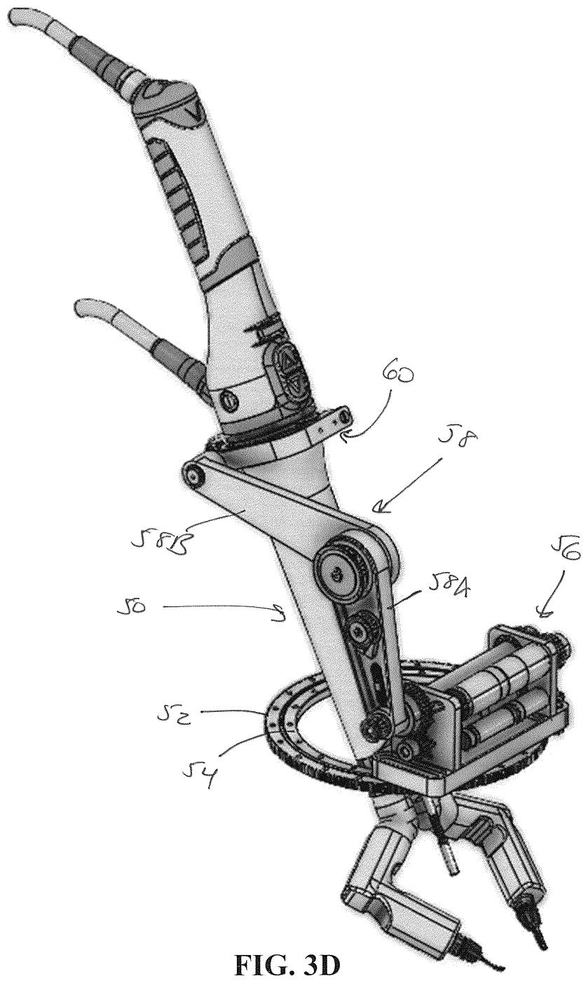

The gross robot positioning device 44 according to one embodiment with a robotic device 50 attached thereto is depicted in additional detail in FIGS. 3A-3D. In this implementation, the device 44 has a first (or outer) ring 52 attached to a support arm (such as arm 42 discussed above) such that the outer ring 52 generally does not rotate, a second (or inner) ring 54 rotatably coupled to the first ring 52, an actuation assembly 56 fixedly attached to the inner ring 54, a rotatable arm 58 (made up of first and second links 58A, 58B) rotatably coupled to the actuation assembly 56, and a device attachment structure (or clamp) 60 rotatably coupled to the rotatable arm 58. Alternatively, in certain embodiments, the inner ring 54 can be attached to the support arm and the outer ring 52 can be rotatable in relation to the inner ring 54.

A surgical robotic device (such as device 50) can be docked or otherwise removably coupled to the gross positioning device 44 via the connecting clamp 60 such that the clamp 60 is removably attached to the body of the device 50 and the body of the device 50 extends through the opening 62 defined in the inner ring 54, as best shown in FIGS. 3A-3D. In this particular embodiment, the clamp 60 is coupleable to the robotic surgical device 44 at a specific location on the device 44 having a recessed area (e.g., a clamping groove) (such as the groove 13 depicted in the device 12 of FIG. 1B) around the external surface of the device 44 such that the clamp 60 can easily be disposed within the recessed area 13. Alternatively, any known coupling feature or mechanism can be used. In this embodiment, when the surgical robotic device 50 is docked with the gross positioning device 44, it does not move or rotate with respect to the clamp 60. The attachment mechanism 60 is easily and quickly disengaged when desired by the user.

As discussed above, the port 46 as best shown in FIG. 2B can come in various forms. One embodiment would be a gelport that includes a gel like substance that would seal around the circumference of the robot, to maintain insufflation, while still allowing the robot to move. Another port would use air flow to maintain patient insufflation. Others might use various types of mechanical seals such as diaphragm, duck bill, O-ring or other types of seals or ports. Any known port that can maintain a fluidic seal can be used.

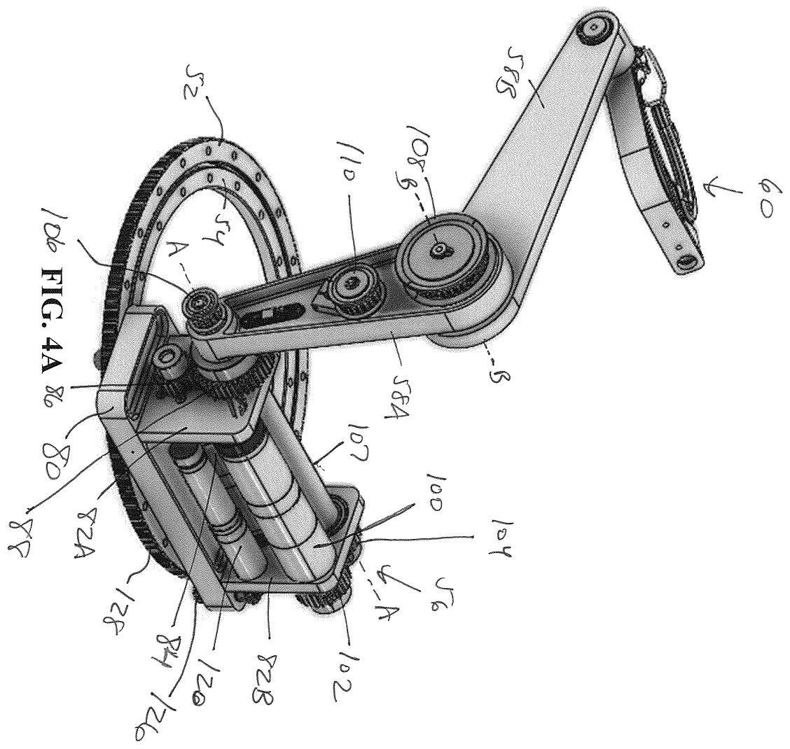

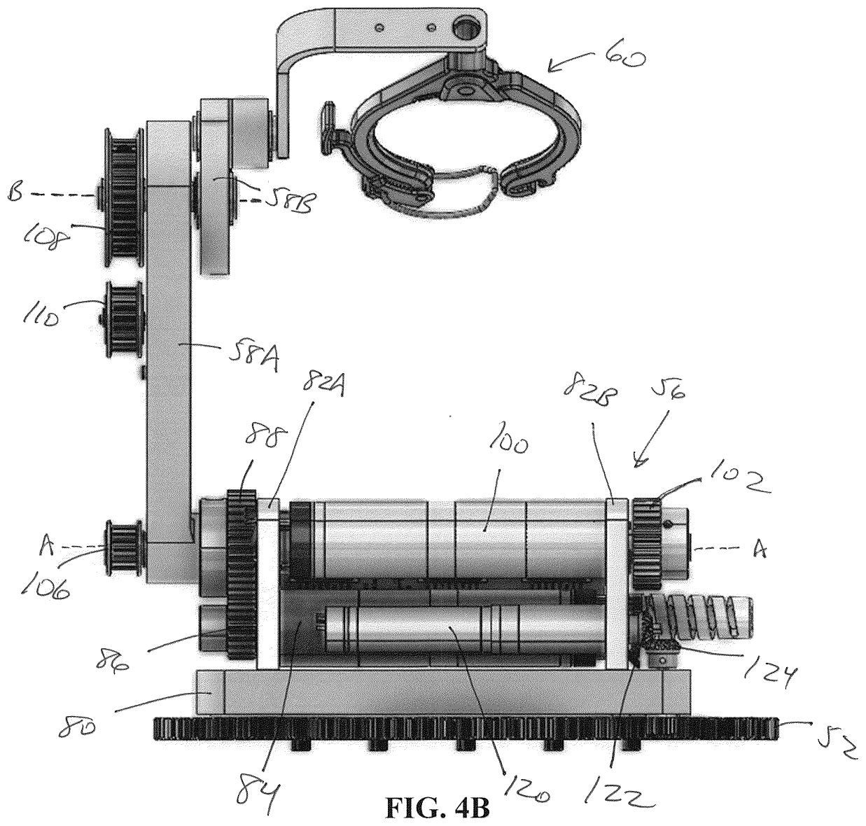

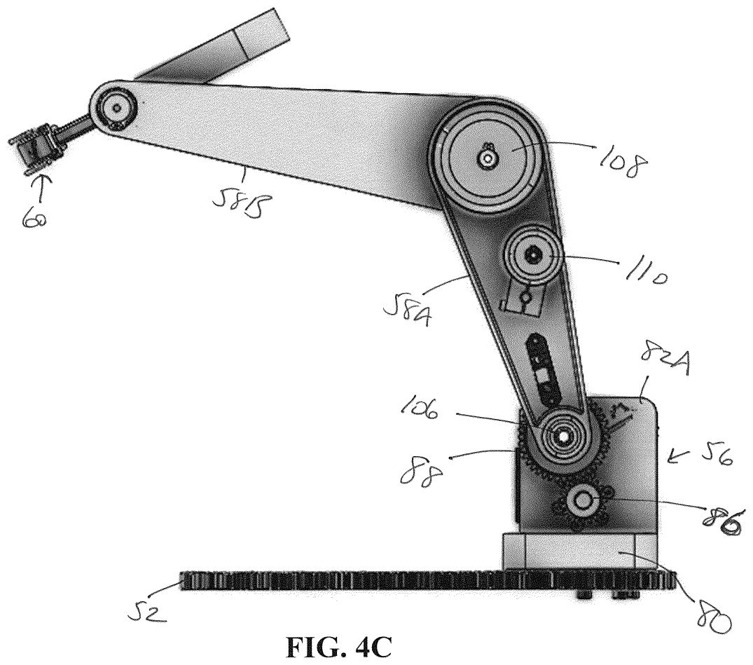

FIGS. 4A-4C depict the gross positioning device 44 without a robotic device coupled thereto. Further, the device 44 as shown in FIGS. 4A-4C has no housings or casings disposed thereon, such that the internal components and couplings of the device 44 are shown.

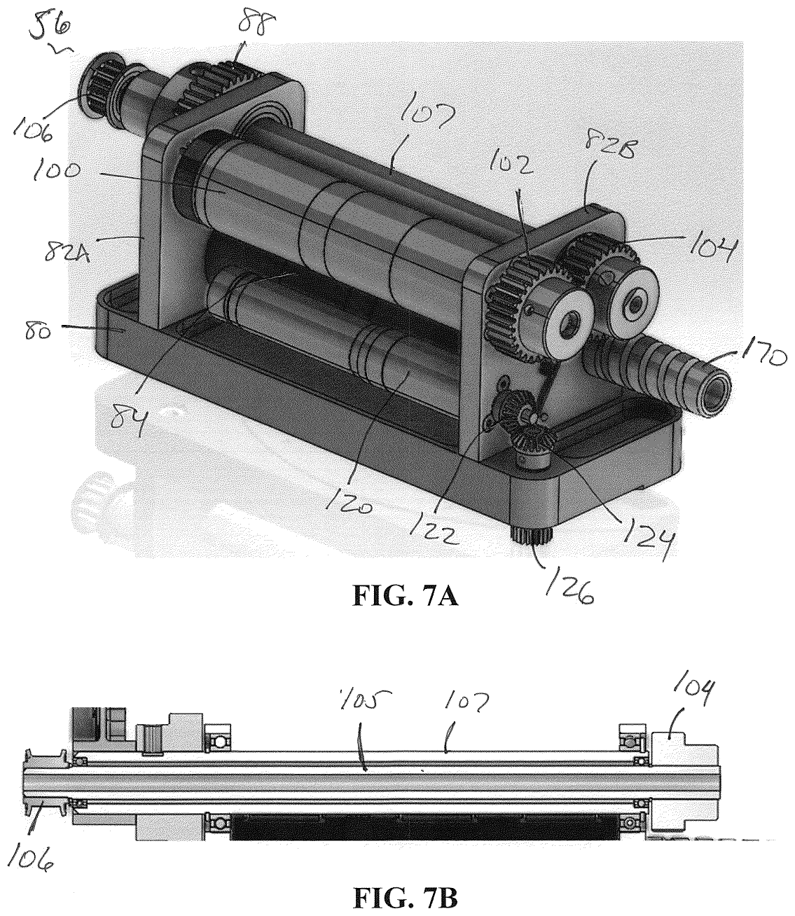

As best shown in FIGS. 4A-4C and 7A-7B, the actuation assembly 56 has a base 80 and two vertical supports 82A, 82B. The base 80 is fixedly attached to the inner ring 54 via an attachment screw 160 as best shown in FIG. 6. That is, the attachment screw 160 is disposed through the inner ring 54 and into the base 80, thereby attaching the base 80 to the inner ring 54. Further, as discussed in further detail below, the base 80 is rotatably coupled to the outer ring 52 via the ring drive gear 126 such that rotation of the gear 126 causes movement of the base 80 in relation to the outer ring 52. Three motors 84, 120, 100 are disposed between and attached to the vertical supports 82A, 82B: the first (or first link) motor 84, the second (or second link) motor 100, and the third (or ring) motor 120.

The first (or first link) motor 84 has a drive gear 86 rotatably coupled to the motor 84, and the drive gear 86 is rotatably coupled to a driven gear 88. The driven gear 88 is fixedly attached (or rotationally constrained) to the first arm link 58A such that actuation of the first motor 84 causes rotation of the first arm link 58A around the axis A.

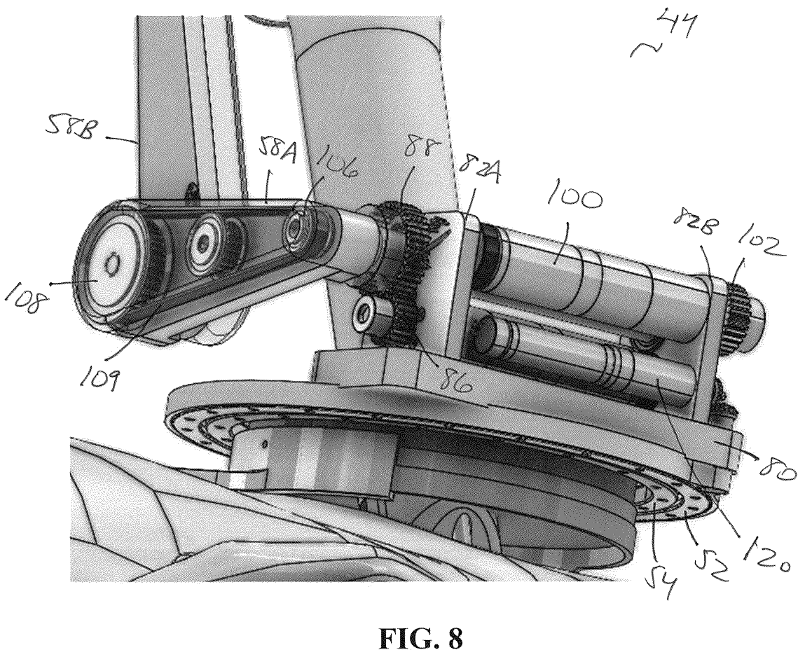

The second (or second link) motor 100 has a drive gear 102 rotatably coupled to the motor 100, and the drive gear 102 is rotatably coupled to a driven gear 104 (as best shown in FIG. 4A). The driven gear 104 is fixedly attached (rotationally constrained) to a driven shaft 105, which is rotatably disposed within the shaft 107 and is fixedly attached (rotationally constrained) to first belt gear 106. A belt 109 or other similar structure is coupled to the first belt gear 106 and extends to the second belt gear 108 (as best shown in FIG. 8) such that rotation of the first belt gear 106 causes rotation of the second belt gear 108 via the belt 109. The second belt gear 108 is fixedly attached (or rotationally constrained) to the second arm link 58B such that rotation of the second belt gear 108 causes rotation of the second arm link 58B around axis B. A tension pulley 110 is attached to the first arm link 58A such that the tension pulley 110 can be used to apply a desired amount of tension to the belt 109. Thus, actuation of the second motor 100 causes rotation of the second arm link 58B around the axis B.

The third (or ring) motor 120 has a drive gear 122 rotatably coupled to the motor 120, and the drive gear 102 is rotatably coupled to a driven gear 124 (as best shown in FIG. 4B). The driven gear 124 is fixedly attached (rotationally constrained) to a ring drive gear 126 (as best shown in FIGS. 4A and 7A), and the drive gear 126 is rotatably (and threadably) coupled to the outer ring 52. Because the actuation assembly 56 is fixedly attached to the inner ring 54 (as described above and depicted in FIG. 6) and the drive gear 126 is threadably coupled to the outer ring 52, rotation of the drive gear 126 causes rotation of the inner ring 54 in relation to the outer ring 52. As such, actuation of the third motor 120 causes rotation of the inner ring 54 in relation to the outer ring 52.

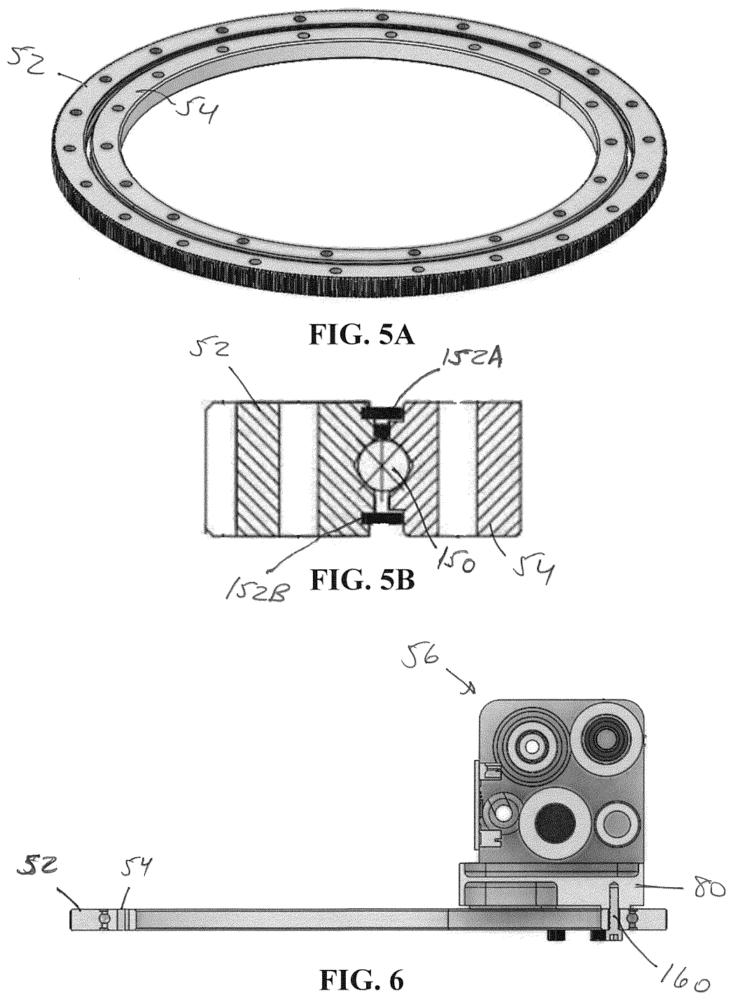

One embodiment of the rings 52, 54 is depicted in FIGS. 5A and 5B. As best shown in FIG. 5B, a bearing 150 is disposed between the outer ring 52 and the inner ring 54 to facilitate rotation of the inner ring 54 in relation to the outer ring 52. Further, two seals 152A, 152B are positioned on either side of the bearing 150 such that a fluidic seal is maintained between the two rings 52, 54 (thereby allowing the rings 52, 54 to establish a fluidic seal between the interior of the patient's cavity and the ambient air outside the patient during a procedure).

FIG. 8, according to one implementation, depicts the underside of the positioning device 44 where the device 44 is coupled to the support arm 42. More specifically, the outer ring 52 is attached to the support arm 42, as discussed in further detail below. The ring 52 can be attached via welding, attachment devices (such as screws, bolts, etc.), or any other known attachment structures, mechanisms, or methods.

In operation, the linkage system made up of motors 84, 100 and links 58A, 58B respectively control both plunge depth and pitch of the robotic surgical device 50. Further, motor 120 and the related drive system control the yaw (rotation) of the robotic surgical device 50 about an axis though the center and normal to the inner 54 and outer ring 52 faces.

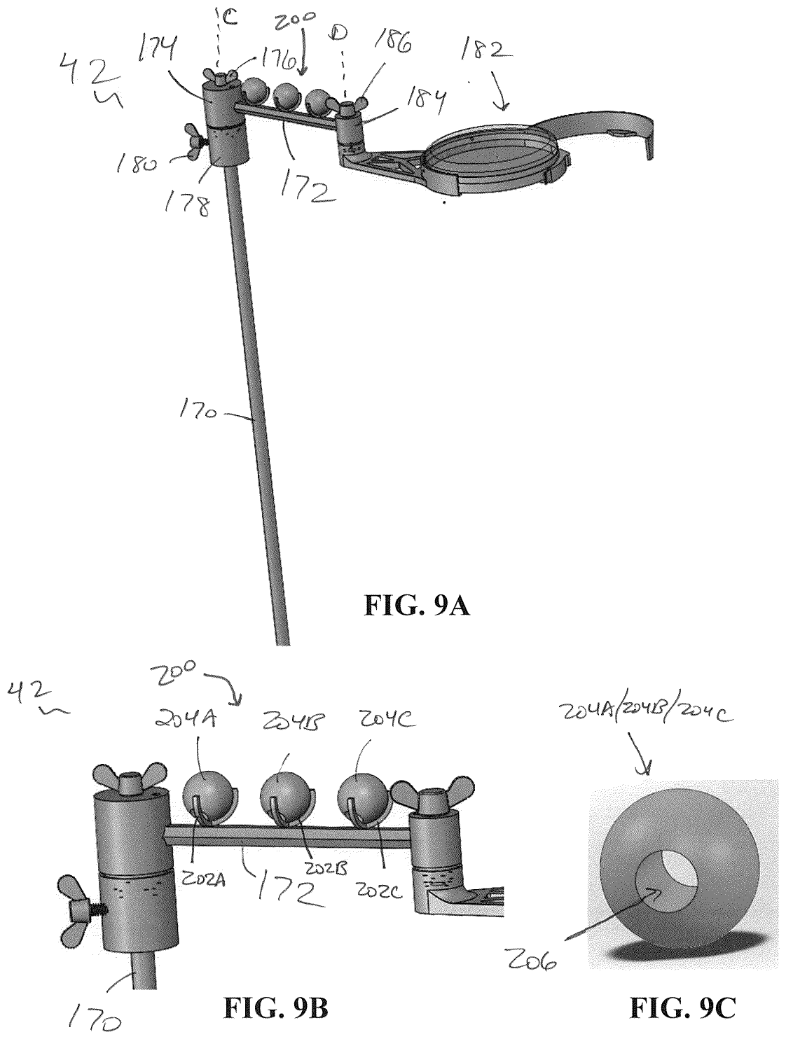

One embodiment of a support arm 42 is depicted in additional detail in FIGS. 9A-9C. The arm 42 has an elongate vertical (or “first”) rod 170 that is coupled at one end (its “bottom” end) to the standard rail of the surgical table (or any known part of the table). At the second (or “top”) end, the rod 170 is rotatably coupled to a horizontal (or “second”) rod 172 at a rotatable joint 174 such that the horizontal rod 172 can rotate around an axis C at the joint 174 that is coaxial with the elongate axis of the vertical rod 172. In one embodiment, the joint 174 is adjustable via a wing nut (or other known locking mechanism or device) 176 that is used to lock or release the joint 174 to allow for adjustment. Further, a lockable height adjustment mechanism 178 can be provided on the vertical rod 170 such that a wing nut (or other known similar locking mechanism or device) 180 can be released to allow for height adjustment of the horizontal rod 172 in relation to the vertical rod 170. That is, the position of the horizontal rod 172 along the length of the vertical rod 170 can be adjustable via the height adjustment mechanism 178.

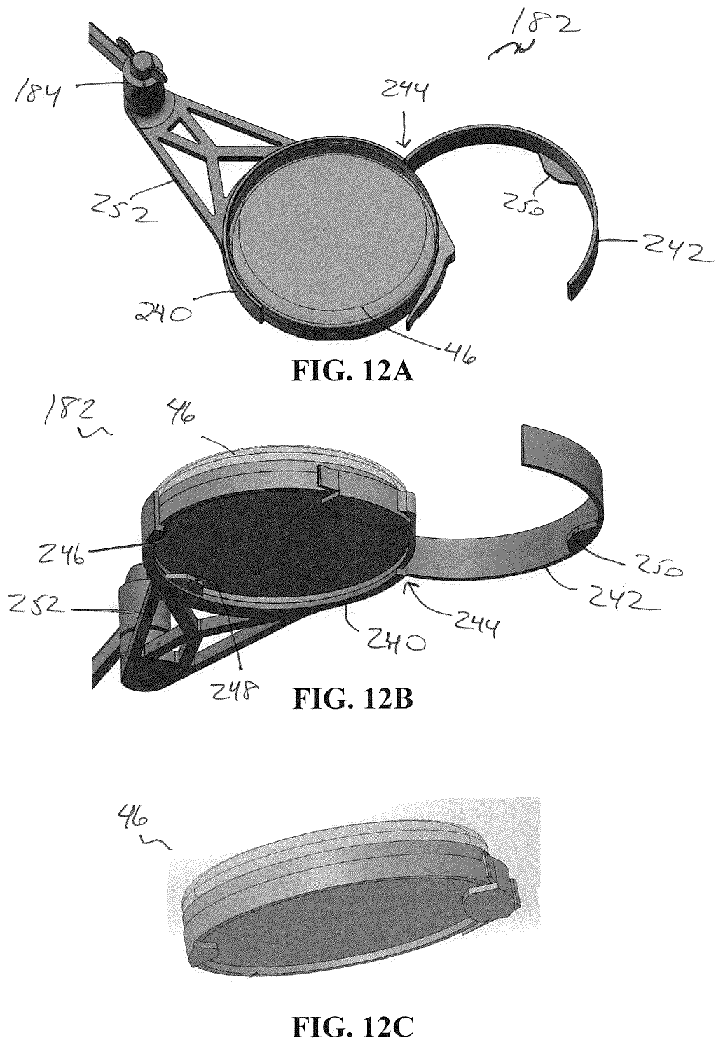

At one end, the horizontal rod 172 is rotatably coupled to the vertical rod 170 via the rotatable joint 174, as discussed above. At the other end, the horizontal rod 172 is rotatably coupled to an attachment mechanism or device (or “clamp”) 182 that can be used to attach the support arm 42 to an incision port (such as port 46 discussed above). One embodiment of the clamp 182 is shown in additional detail in FIGS. 12A-12C and discussed in detail below. The rod 172 is rotatably coupled to the clamp 182 at a rotatable joint 184 such that the clamp 182 can rotate around an axis D at the joint 184 that is transverse with the elongate axis of the horizontal rod 172. In one embodiment, the joint 184 is adjustable via a wing nut (or other known locking mechanism or device) 186 that is used to lock or release the joint 184 to allow for adjustment of the position of the clamp 182.

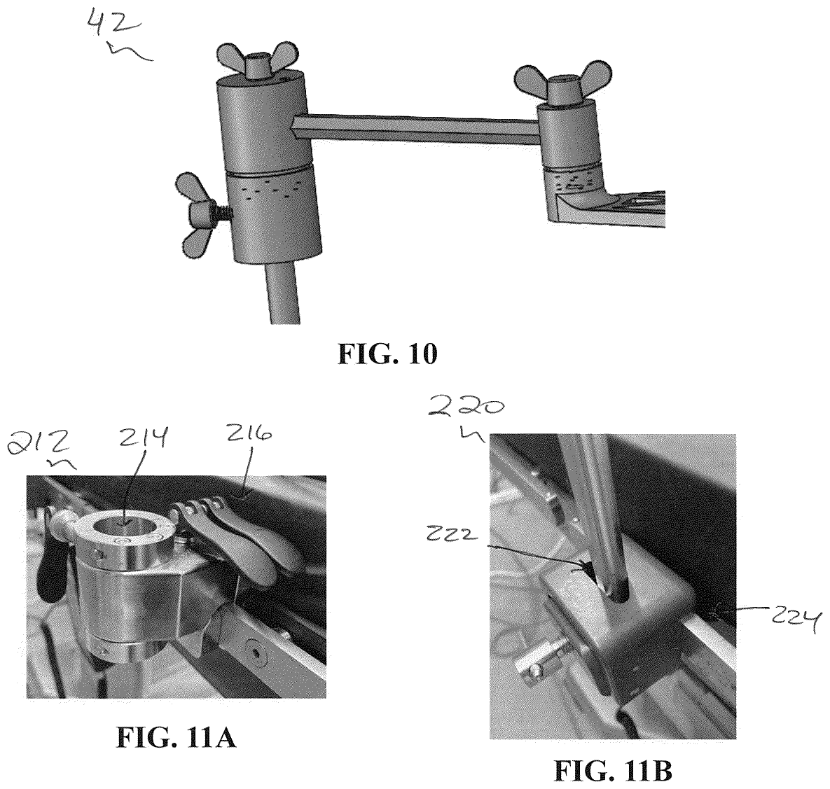

In certain optional implementations, the horizontal arm 172 has a cord attachment structure or mechanism 200 attached thereto. In the specific implementation as shown in FIGS. 9A-9C, the cord attachment structure 200 has three sphere receptacles 202A, 202B, 202C that are configured to receive cord attachment spheres 204A, 204B, 204C. As shown in FIG. 9C, each sphere 204A/B/C has a lumen 206 defined therethrough that is sized to receive a cord (not shown) such that the cord can be inserted through the lumen 206. Thus, each sphere 204A/B/C can be positioned over or molded onto a cord and then placed in one of the receptacles 202A, 202B, 202C such that the positioning of the cord of interest in relation to the surgical space can be controlled. More specifically, in certain embodiments, the cord attachment structure 200 can be used to retain the positioning device cord, the surgical robotic device cord, and the camera cord in position within the sterile environment of the surgical space. Further, according to certain embodiments, the cord attachment structure 200 helps to avoid entanglement of the cords while the positioning device (such as device 44 herein, for example) is used to rotate and otherwise position the surgical robotic device (such as device 12 herein, for example). Alternatively, any known cord retention or attachment structures or mechanisms 200 can be used. In a further alternative, the support arm 42 does not have any cord attachment mechanism or structure, as shown in FIG. 10.

As discussed above, the bottom end of the vertical rod 170 is attached to a standard railing of the surgical table. As shown in FIGS. 11A and 11B, various attachment devices can be used to attach the rod 170 to the rail 210. More specifically, FIG. 11A depicts an attachment device 212 that has a lumen 214 to receive the rod 170 and a rail attachment device 216. Alternatively, FIG. 11B depicts a known attachment device 220 that has a lumen 222 to receive the rod 170 and a rail attachment device 224. Alternatively, any known railing attachment device can be used.

One exemplary embodiment of the port attachment clamp 182 is depicted in FIGS. 12A-12C. The clamp 182 has a ring body 240 and a rotatable clamp arm 242 that is rotatably attached at one end to the ring body 240 via the rotatable joint 244. As such, the arm 242 can rotate between an open position as shown in FIGS. 12A and 12B and a closed or clamped position as shown in FIG. 12C. As best shown in FIG. 12B, the ring body 240 has a lip 246 extending from a lower edge of the body 240 that is configured to receive the incision port (such as port 46) such that the port sits on the lip 246. Further, the body 240 can also have a protrusion 248 extending from the lip 246 that further helps to retain the port 46 within the clamp 182. In addition, the clamp arm 242 can also have a similar protrusion 250 to serve the same purpose when the arm 242 is in the closed position. The clamp 182 also has an extension body or arm 252 attached on one side to the ring body 240 and at the other side or end to the rotatable joint 184.

In use, the clamp body 240 is positioned around the target port (such as port 46) such that the port is disposed within the body 240 and in contact with the lip 246. At this point, the arm 242 is urged into its closed position (and attached to the ring body 240 in that closed position) such that the clamp 182 is attached to the port.

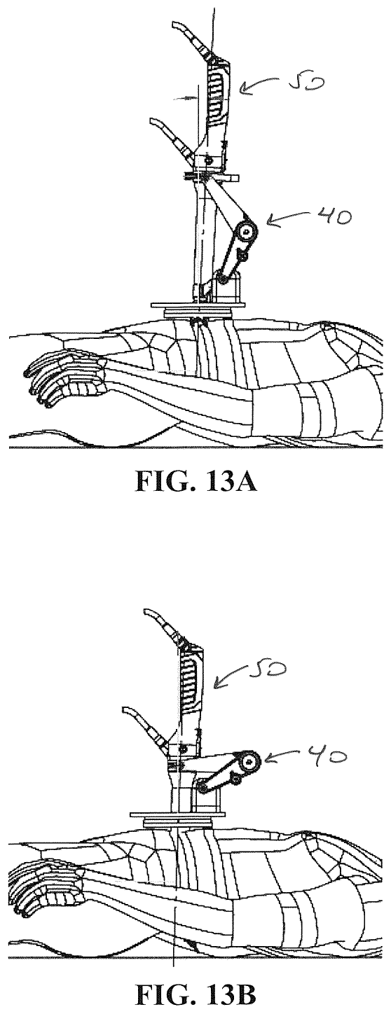

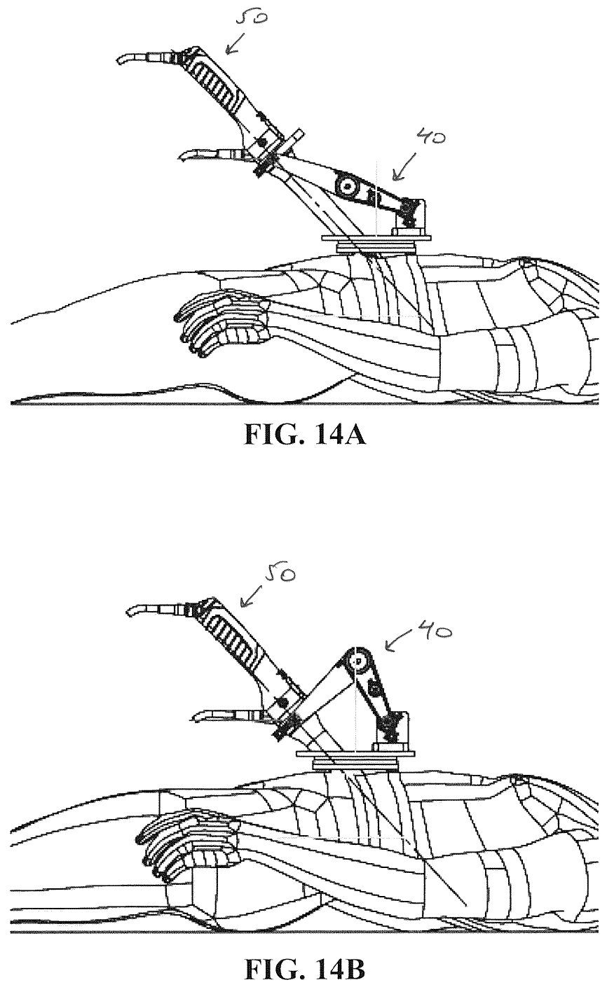

In accordance with certain embodiments as shown in FIGS. 13A-14B, any of the positioning device implementations herein can precisely position the robotic device 50 though the incision port at a certain height in relation to the ring assembly. For example, when the device 50 is disposed at a substantially vertical position as shown in FIGS. 13A and 13B, the positioning system 40 can move the device 50 between a minimum plunge position (in which the proximal end of the device 50 is positioned at a maximum distance from the ring assembly) as shown in FIG. 13A and a maximum plunge position (in which the proximal end of the device 50 is positioned a minimum distance from the ring assembly) as shown in FIG. 13B. Similarly, when the device 50 is disposed at an angled position as shown in FIGS. 14A and 14B, the positioning system 40 can move the device 50 between a minimum plunge position (in which the proximal end of the device 50 is positioned at a maximum distance from the ring assembly) as shown in FIG. 14A and a maximum plunge position (in which the proximal end of the device 50 is positioned a minimum distance from the ring assembly) as shown in FIG. 14B.

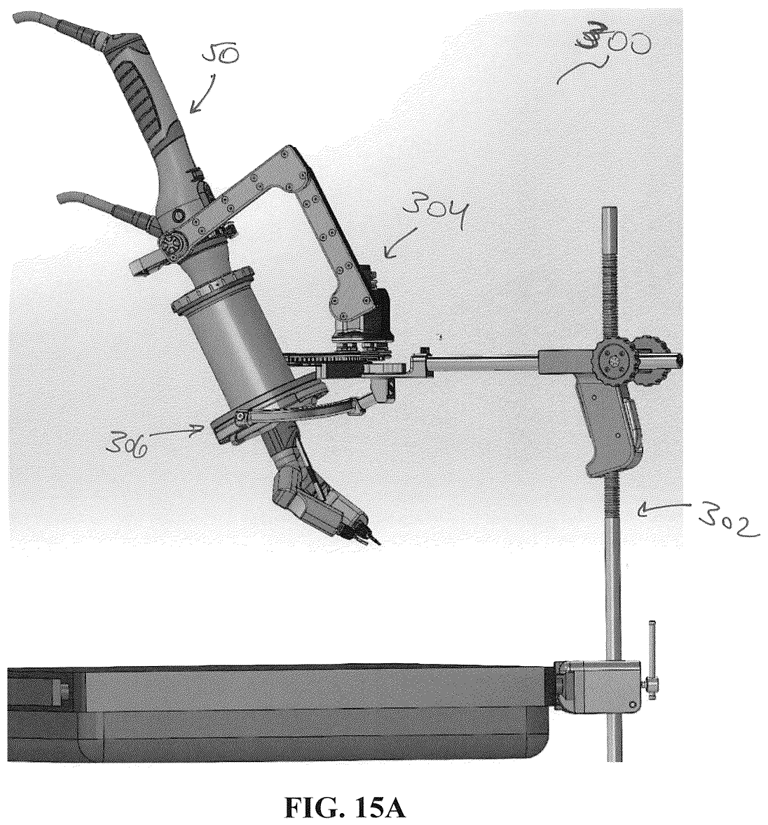

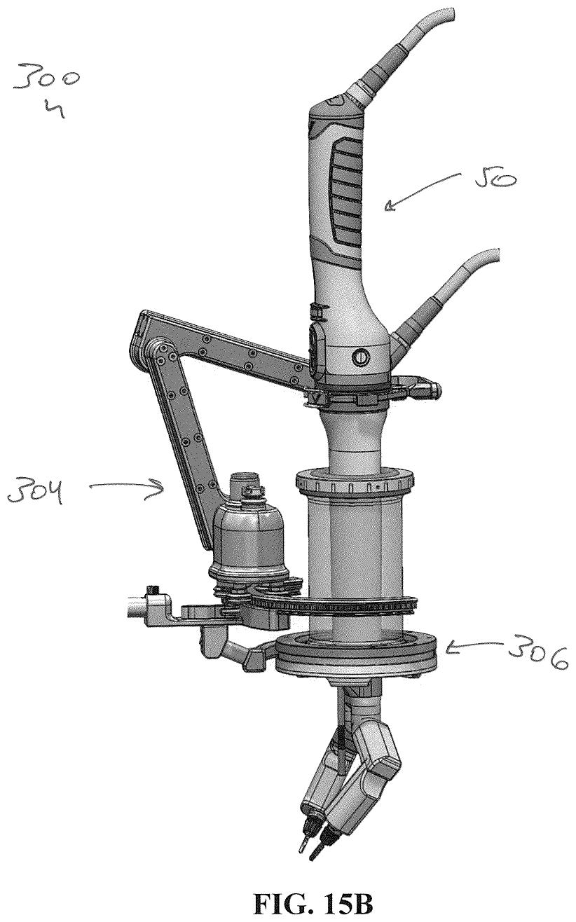

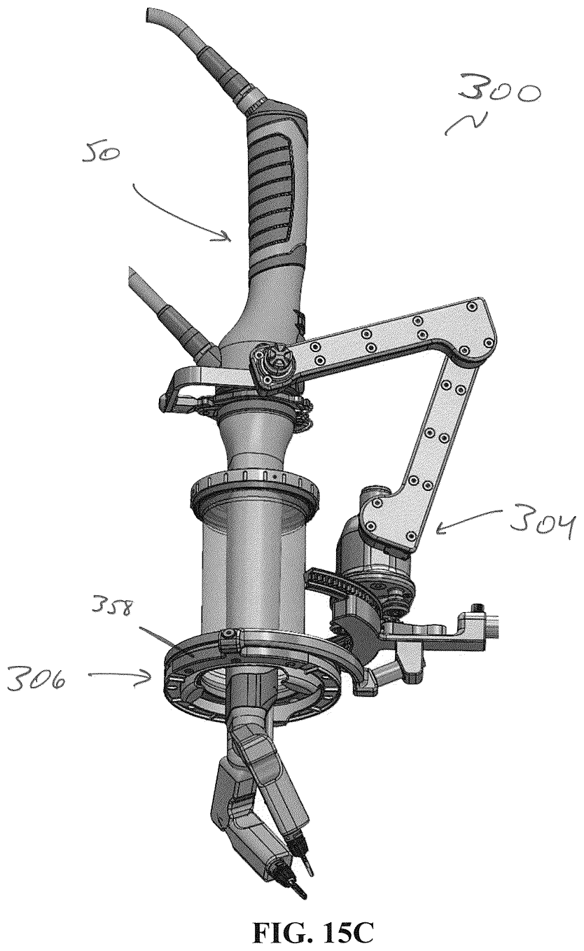

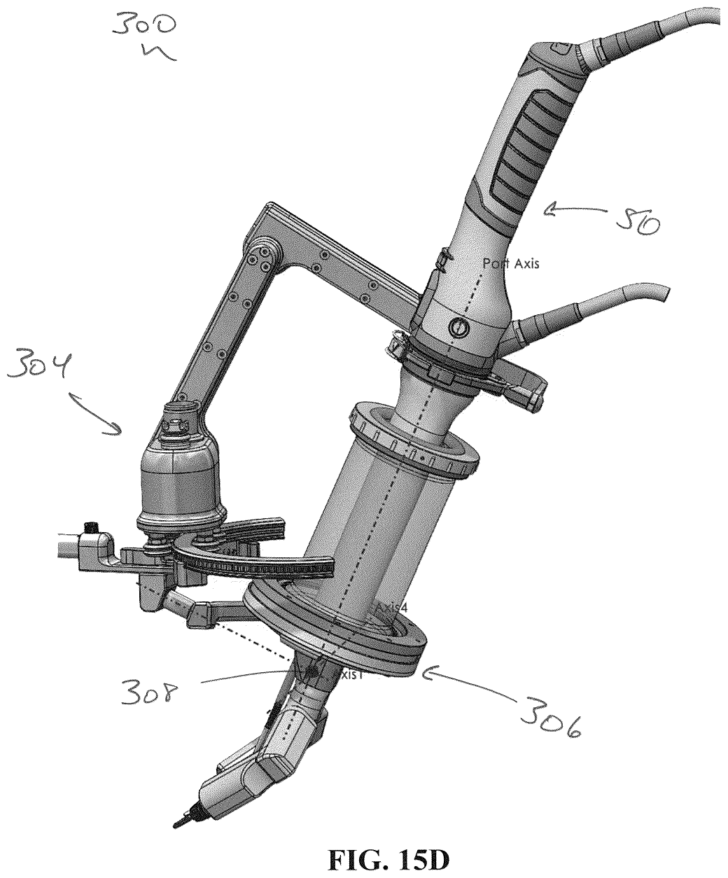

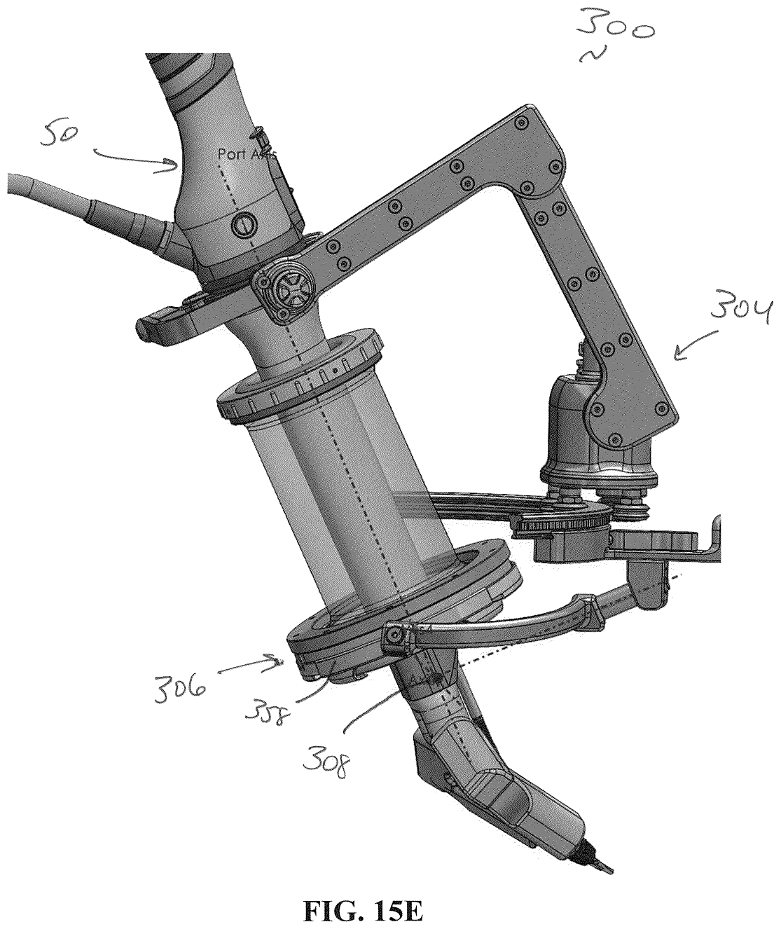

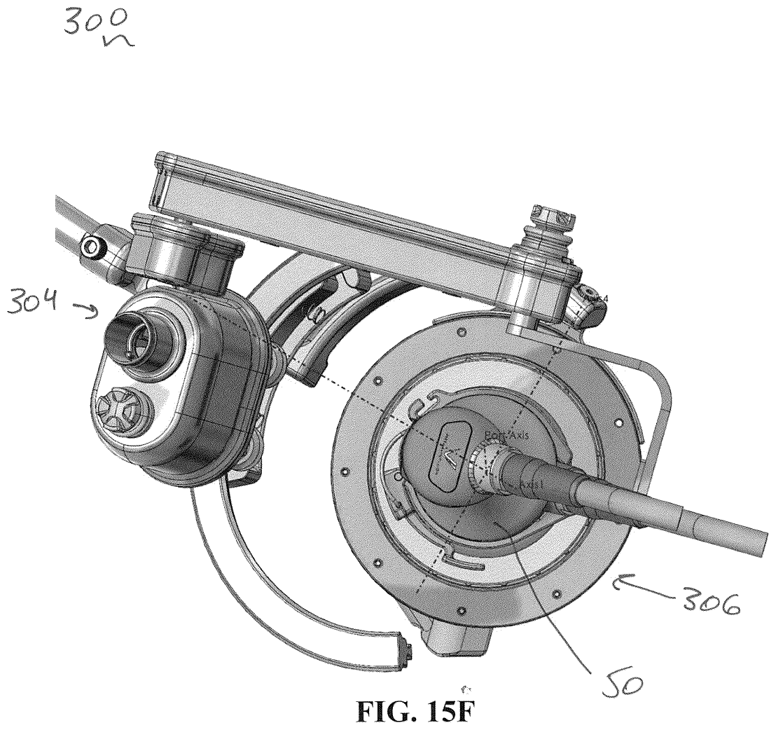

Another implementation of a robotic surgical device positioning system 300 is depicted in FIGS. 15A-15F. The system 300 includes a passive support arm 302 and a gross robot positioning device 304 coupled to the arm 302. As will be described in further detail below (and in contrast to the support arm 42 embodiment above), the passive support arm 302 is coupled to the gross robot positioning device 304 and the gross robotic positioning device 304 is coupled to a port 306. Any known robotic device 50, which in this specific exemplary implementation is represented by the surgical device 50 as shown, can be removably coupled to the positioning device 304 such that the robotic surgical device 50 is disposed through the port 306 into the target cavity of the patient.

Like the device 44 above, the gross positioning device 304 according to this implementation is a 3 degree-of-freedom (“DOF”) robotic remote center-of-motion (RCM) mechanism, controlling yaw, pitch and plunge of the robotic surgical device 50. In this specific embodiment as will be discussed in additional detail below (and as best shown in FIGS. 15D-E), the RCM 308 is located not above the incision (essentially at the outer skin level) but within the incision in the cavity wall (such as the abdominal wall) of the patient—some distance below the portion of the port that is positioned on the outer skin of the patient. As such, there is no relative motion at this patient-device interface at the RCM 308 that could cause harm to the patient, while still allowing the robotic surgical device 50 full access to the target surgical site (a cavity within the patient).

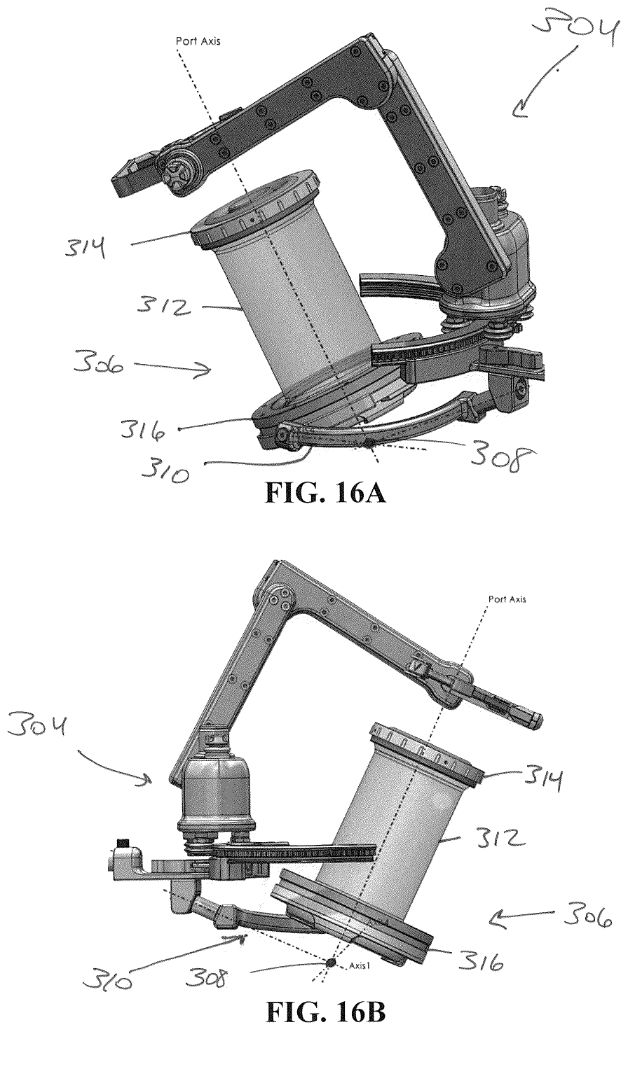

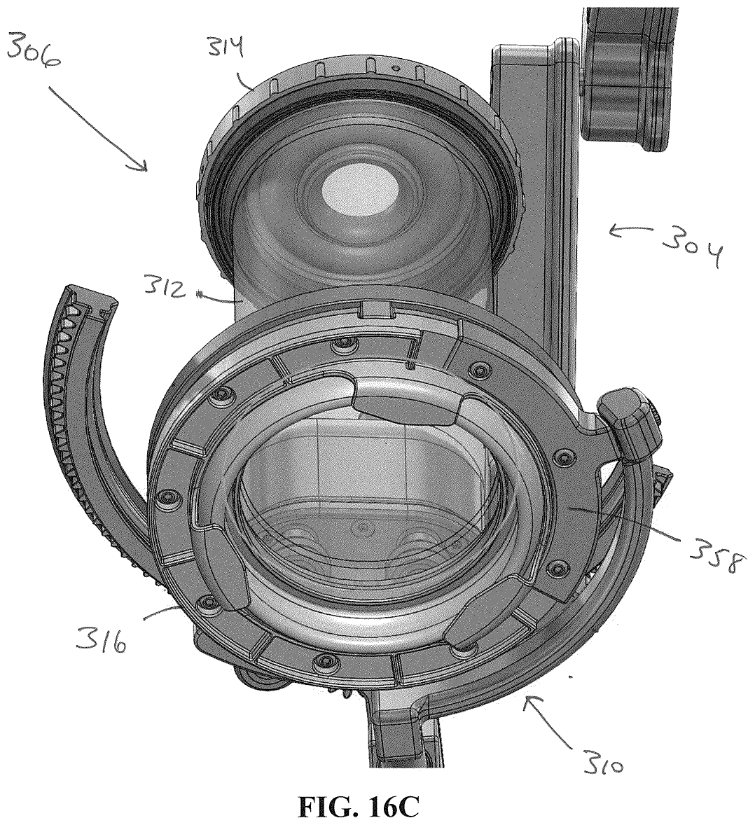

One embodiment of the gross robot positioning device 304 with the flexible port 306 attached thereto is depicted in additional detail in FIGS. 16A-16C. More specifically, the positioning device 304 has a port support structure 310 attached thereto that is disposed below the rest of the positioning device 304 and to which the flexible port 306 is attached. In this exemplary implementation, the flexible port 306 has a flexible tubular body 312 with a proximal ring 314 at a proximal (or top) end of the body 312 and a distal ring 316 at a distal (or bottom) end of the body 312 with a lumen defined through the port 306 such that a robotic device (such as device 50) can be positioned therethrough as best shown in FIGS. 15A-15F. In one specific embodiment, the flexible port is disclosed in further detail in U.S. patent application Ser. No. 18/516,078, filed on Nov. 21, 2023 and entitled “Insertion and Access Device for Surgical System,” which is hereby incorporated herein by reference in its entirety. Alternatively, the port can be any commercial-available port or trocar for use in various procedures through an incision, such as laparoscopic surgery or the like. One embodiment would be a commercially available gelport that includes a gel-like substance that would seal around the circumference of the robot, to maintain insufflation, while still allowing the robot to move. Another port embodiment uses air flow to maintain patient insufflation. Others might use various types of mechanical seals such as diaphragm, duck bill, O-ring or other types of seals or ports. Alternatively, any known port that can maintain a fluidic seal can be used.

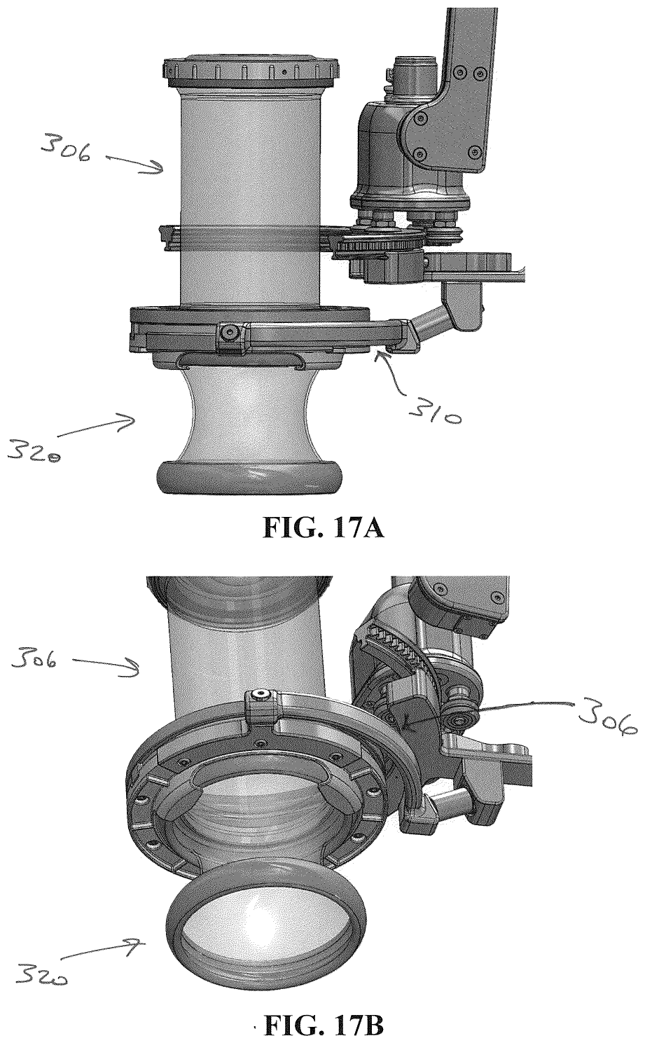

In certain embodiments as depicted in FIGS. 17A-17B, the flexible port 306 is configured to be coupleable with a commercially-available wound protector (or retractor) 320 such that the wound protector is disposed through the incision during use. In one specific implementation, the wound protector is the Wound Protector SurgiSleeve™ 2.5 to 6 cm, which is manufactured by Medtronic MITG (https://mms.mckesson.com/product/860691/Medtronic-MITG-WPSM256). Alternatively, any standard wound retractor or protector can be used. In use, the bottom ring 316 of the flexible port 306 is coupled to the top ring of the wound retractor 320 such that the wound retractor is disposed through the incision in the cavity wall of the patient. Further details about certain, non-limiting embodiments of the wound retractor 320 and the flexible port 306 are described in U.S. patent application Ser. No. 18/516,078, which is incorporated by reference above.

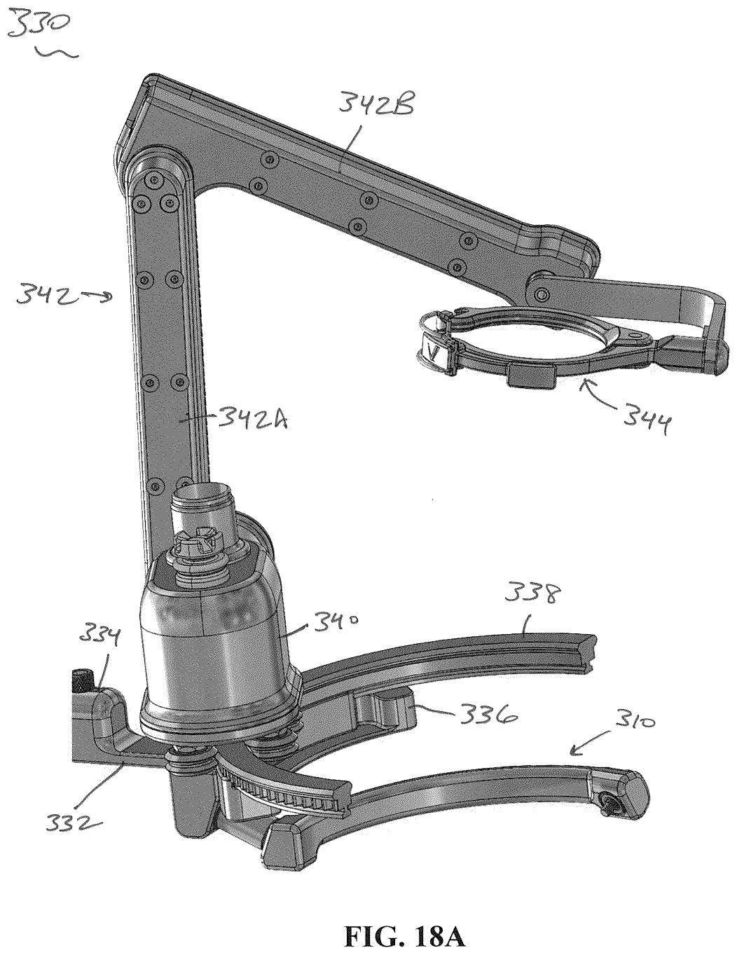

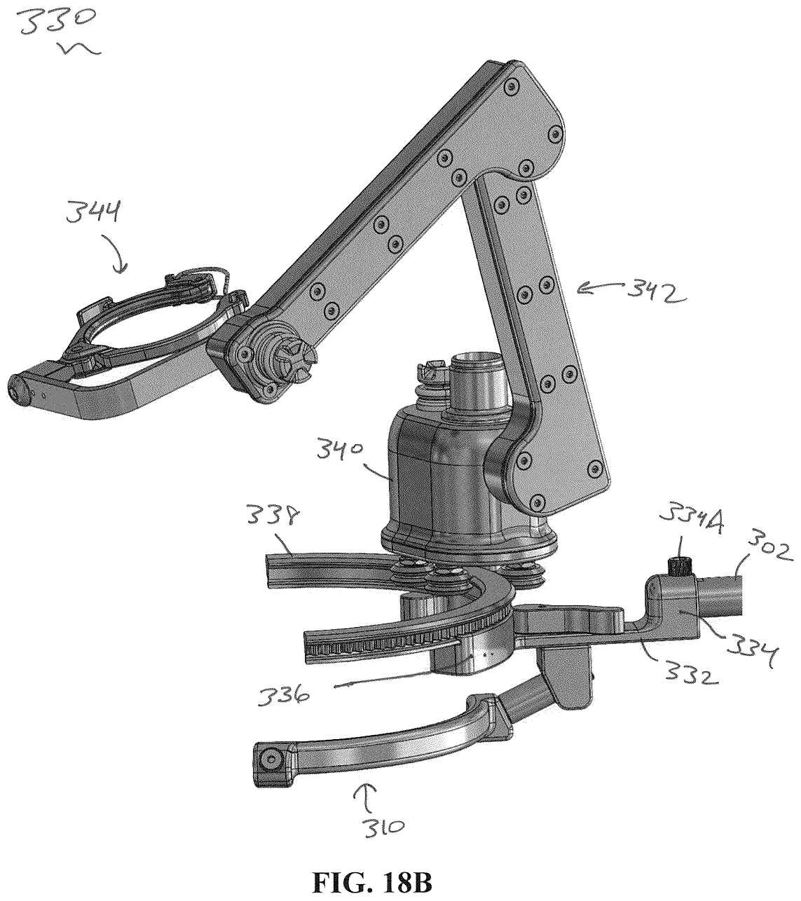

One exemplary embodiment of the gross robot positioning device 330 without a robotic device attached thereto is depicted in additional detail in FIGS. 18A-18B. In this implementation as best shown in FIG. 18B, the device 330 has a device base 332 with a support arm attachment component 334 at a proximal end and a ring attachment component 336 at a distal end of the base 332. Further, the device 330 has a positioning ring 338 attached to the ring attachment component 336 and an arm base (also referred to as an “actuation assembly”) 340 movably attached to the positioning ring 338. In addition, the device 330 has a positioning arm 342 rotatably coupled to the arm base 340, with the arm 342 being made up of two arm links 342A, 342B, with a device attachment structure (or clamp) 344 rotatably coupled to the positioning arm 342 as will be described in additional detail below. As mentioned above, the device 330 also has a port support structure 310 that is attached to the device base 332 and attaches to the port or trocar during use, as will also be described in further detail below.

In one embodiment, the support arm attachment component 334 is configured to removably couple to the horizontal rod of the support arm 302 such that the positioning device 330 is coupled to the passive support arm 302 as best shown in FIG. 18B. More specifically, the attachment component 334 can be made up of an opening (not shown) in the proximal end of the attachment component 334 that is sized to receive the distal end of the horizontal rod of the support arm 302 such that the attachment component 334 is positioned over the rod. Further, the component 334 can also have a tightening screw 334A that is rotatably positioned through a threaded lumen (not shown) that extends into the opening (not shown) such that the tightening screw 334A can be tightened into contact with the distal end of the rod such that the tightening screw 334A retains the rod within the opening. Alternatively, the support arm attachment component 334 can be any known component, mechanism, or feature for attaching the device 330 to the passive support arm 302.

A surgical robotic device (such as device 50) can be docked or otherwise removably coupled to the gross positioning device 330 via the connecting clamp 344 such that the clamp 344 is removably attached to the body of the device 50 and the body of the device 50 extends through an area adjacent to the positioning ring 338 and through a port (such as port 306 described above, for example) attached to the port support structure 310, as best shown in FIGS. 15A-15F. In this particular embodiment, the clamp 344 is coupleable to the robotic surgical device (such as either of devices 12 or 50) at a specific location on the device having a recessed area (e.g., a clamping groove) (such as the groove 13 depicted in the device 12 of FIG. 1B) around the external surface of the device 50 such that the clamp 344 can easily be disposed within the recessed area 13. Alternatively, any known coupling feature or mechanism can be used. In this embodiment, when the surgical robotic device (such as device 12 or 50) is docked with the gross positioning device 330, it does not move or rotate with respect to the clamp 344. The attachment mechanism 344 is easily and quickly disengaged when desired by the user.

In use, once the robotic device (such as device 50) is attached to the positioning device 330 as shown above with respect to FIGS. 15A-15F, the positioning device 330 can be actuated to precisely position the robotic device 50, and more specifically to position the distal end of the device, including the arms and end effectors, within the cavity of the patient while maintaining the RCM 308 within the incision of the patient. That is, the movable arm base 340 can move along the positioning ring 338 as will be described in detail below and the arm 342 can be rotated via one or both links 342A, 342B to urge the proximal portion of the robotic device (such as device 50) into the desired position to ensure that the distal portion of the device, including the arms and end effectors, are positioned as desired in the patient cavity.

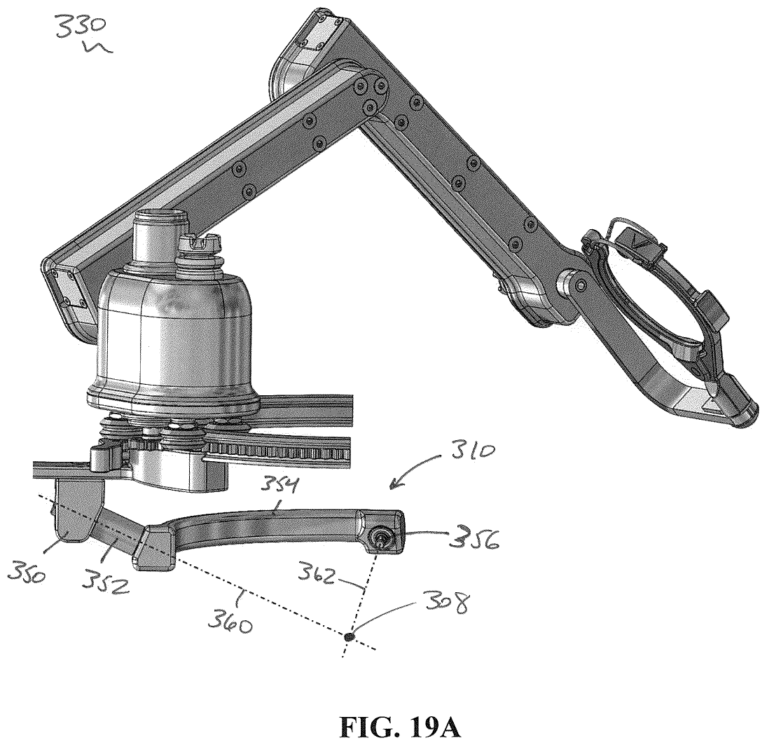

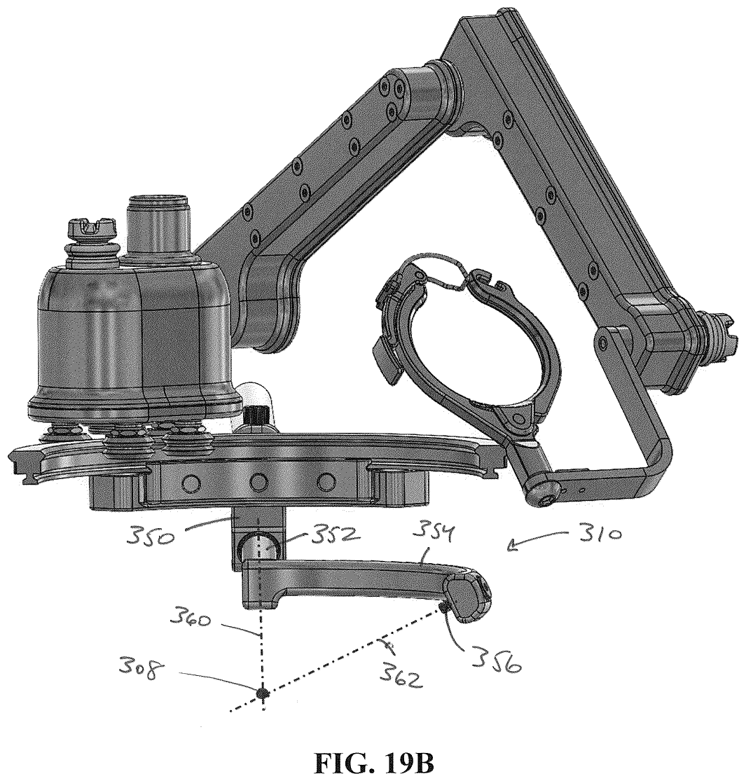

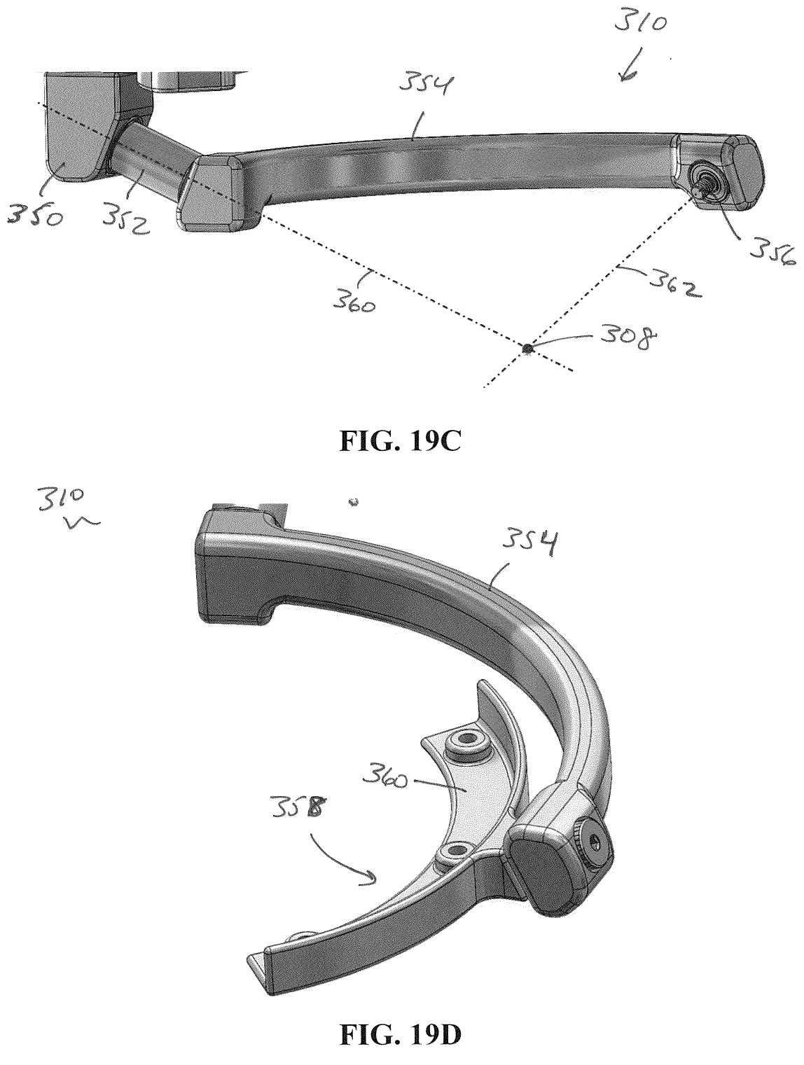

One specific implementation of a port support structure 310 is depicted in additional detail in FIGS. 19A-19D. It is the port support structure 310 that makes it possible for the remote center-of-motion 308 of the positioning device 330 to be disposed within the incision in the patient's cavity wall (rather than at the outer skin surface of the patient). The support structure 310 has a base body 350 that is attached to an underside of the device base 332, with a connection rod 352 extending from the base body 350 at a predetermined angle. In one exemplary embodiment, the connection rod 352 is disposed at an angle of about 24 degrees in relation to the device base 332. Alternatively, the angle can range from about zero degrees to about 45 degrees in relation to the device base 332. In a further alternative, the angle can range from about 10 to about 35 degrees. Coupled to the rod 352 is the semicircular arm 354, which is substantially parallel with the device base 332 and has a rotational joint 356 at the end of the arm 354 opposite the rod 352 with a rotational axis 362 that extends from the arm 354 at an angle of about 26 degrees in relation to the arm 354. Alternatively, the angle can range from about zero degrees to about 45 degrees in relation to the arm 354. In a further alternative, the angle can range from about 10 to about 35 degrees. And as best shown in FIG. 19D, the support structure 310 has a port attachment structure 358 rotatably coupled to the rotational joint 356, thereby resulting in a rotatable port attachment structure 358. The attachment structure 358 can be coupled to any port or trocar for use with the positioning device 330 as discussed elsewhere herein. In one exemplary embodiment, the attachment structure 358 is a curved structure 358 with a lip 360 on which the port or trocar can be positioned. For example, FIGS. 15C, 15E, and 16C depict the attachment structure 358 coupled to the distal ring 316 of the flexible port 306, in accordance with one implementation.

It is the location of the rotational axis 362 of the rotational joint 356 in relation to the elongate axis 360 of the connection rod 352 that determines the position of the RCM 308 in the positioning device 330. More specifically, the RCM 308 is the point of intersection of the elongate axis 360 of the connection rod and the rotational axis 362 of the rotational joint 356, as shown in FIGS. 19A-19C. As such, the length and specific curvature of the semicircular arm 354 are predetermined to ensure that the rotational axis 362 intersects with the elongate axis 360 at the desired location in relation to the port (such as port 306) that is attached to the attachment structure 358.

In other words, it is the angle of the connection rod 352 and the rotational axis 362 as discussed above that determines the location of the remote center-of-motion 308. For example, in the specific embodiment above in which the connection rod 352 is disposed at an angle of about 24 degrees and the rotational axis 362 is disposed at an angle of about 26 degrees, the RCM 308 is disposed at about 20 mm below the external surface of the abdominal wall of the patient (which is disposed at about the bottom surface of the port (not shown) attached to the port support structure 310). Further, the ranges of the angles above being from about 0 degrees to about 45 degrees results in an RCM that can range from about 0 mm to about 80 mm distance below the external surface of the abdominal wall. It is understood that the angles as discussed above are dependent on certain dimensions of the device 330, including the size of the ring 338, the length of the connection rod 352, the location of the base body 350, etc. As a result, it is also understood that the angles can be adjusted based on those device 330 dimensions to ensure that the RCM is disposed at a desired depth in the range from about 0 mm to about 80 mm below the external surface of the abdominal wall.

Once the RCM 308 is established by the two axes 360, 362, as best shown in FIGS. 15D and 15E, the positioning device 330 can be used to position the robotic device attached thereto (such as device 50, for example) such that the elongate body of the robotic device is disposed through the port 306 attached to the port support structure 310 and thus through the RCM 308, thereby resulting in a portion of the robotic device that remains stationary during movement of the device as explained above. As discussed above, the RCM 308 is disposed within the incision in the patient, thereby resulting in the device body being able to rotate around that point 308 within the incision.

The coupling of the attachment structure 358 to the semicircular arm 354 at the rotational joint 356 results in passive rotation of the attachment structure 358 (and thus the port—such as port 306—attached thereto). Thus, when a robotic device (such as device 50) is attached to the gross positioning device 330 and the positioning device 330 is used to position the robotic device as desired, the positioning arm 342 can urge the proximal portion of the robotic device to move while a distal portion of the device remains stationary at the RCM (within the port—such as port 306). This movement causes the port (and the attachment structure 358 attached thereto) to passively rotate around the rotational axis 362 as a result of the rotational joint 356.

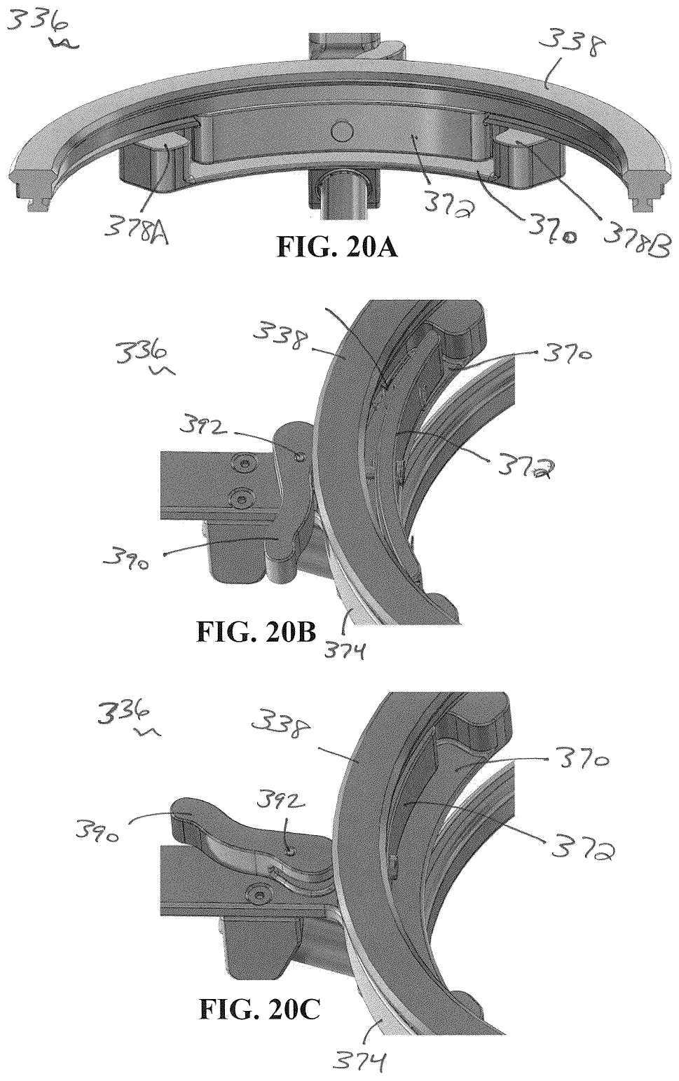

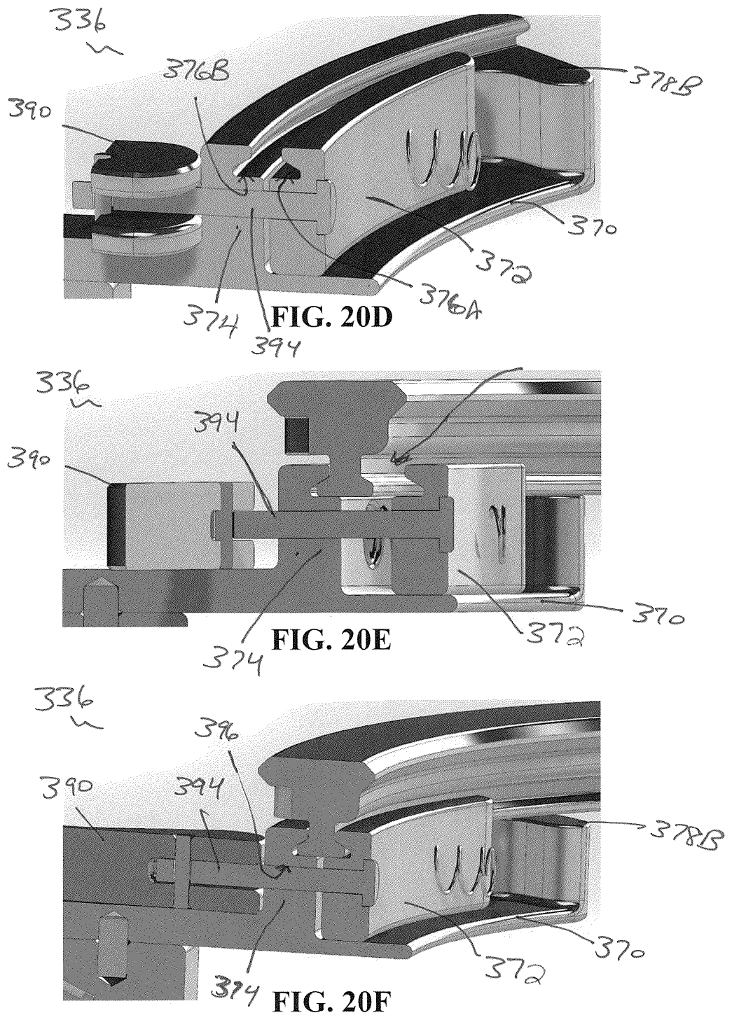

FIGS. 20A-20F depict one embodiment of the ring attachment component 336 that allows for removable attachment of the positioning ring 338 to the positioning device 330. More specifically, the attachment mechanism 336 has a mechanism body 370 with a movable clamping body 372 moveably disposed on the mechanism body 370 such that the clamping body 372 can be moved between an open position (as shown in FIGS. 20B and 20E) and a clamped position (as shown in FIGS. 20C and 20F). As best shown in FIGS. 20D-20E, the moveable clamping body 372 has a channel 376A defined along the length of the body 372 that corresponds to and matches with the clamping rail 378 on the underside of the positioning ring 338 such that a portion of the rail 378 can fit within the channel 376A. Similarly, the stationary wall 374 also has a channel 376B defined along a length of the wall 374 that is substantially a mirror image of the channel 376A such that the channel 376B also corresponds to and matches with the clamping rail 378 such that a portion of the rail 378 can fit within the channel 376B.

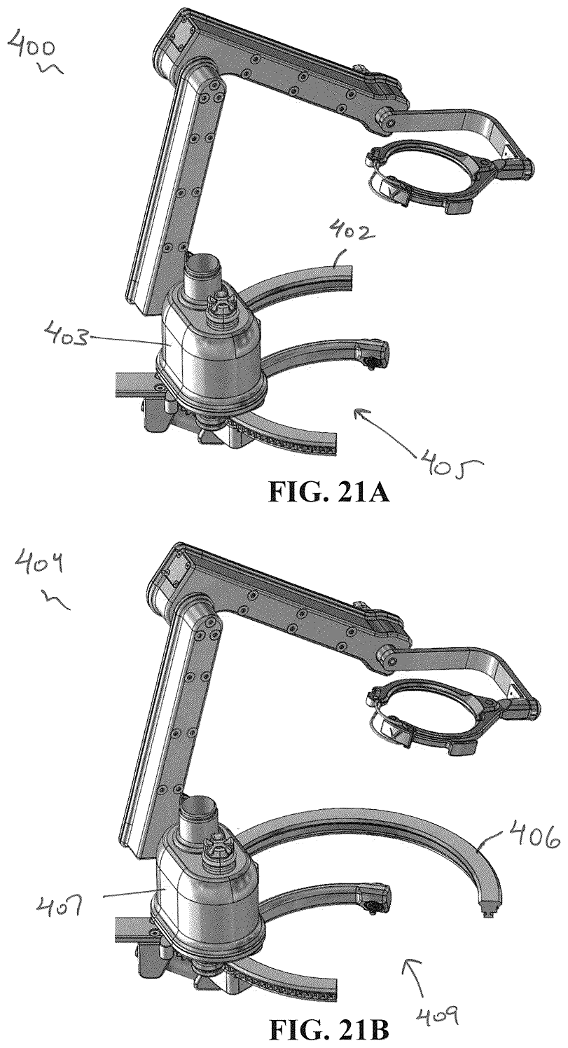

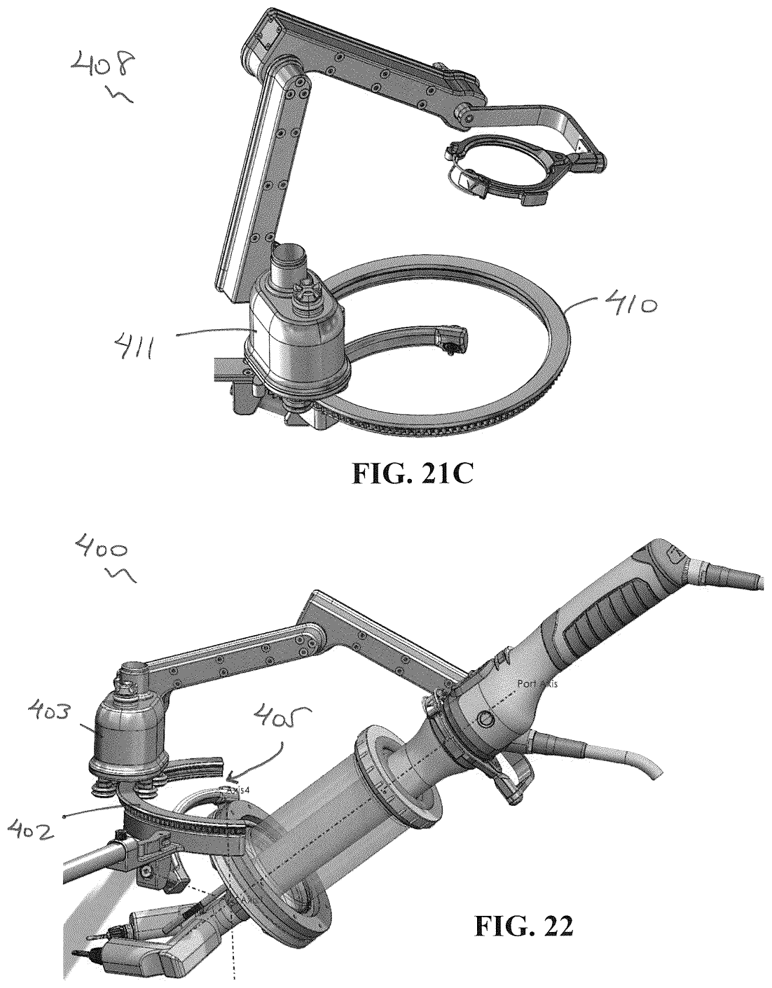

In the open position (as shown in FIGS. 20B and 20E), the clamping body 372 is positioned such that there is a sufficient gap 398 (as best shown in FIG. 20E) between the clamping body 372 and the stationary wall 374 such that the rail 378 of the positioning ring 338 can be easily positioned within the gap 398 between the stationary wall 374 and the clamping body 372. In certain embodiments, the positioning of the positioning ring 338 into the gap 398 for clamping to the positioning device 330 can be facilitated by two shoulders 378A, 378B extending outward on either side of the mechanism body 370. That is, the two shoulders 378A, 378B are disposed to receive and help to position the positioning ring 338 as it is urged into the gap 398. Once the rail 378 is disposed in the gap 398, the clamping body 372 can be urged toward and into contact with or close proximity to the stationary wall 374 such that the rail 378 is clamped or otherwise held in a stable fashion between the clamping body 372 and the stationary wall 374 within the corresponding channels 376A, 376B. This clamping action is a quick process that establishes a secure coupling between the positioning ring 338 and the ring attachment component 336. Further, the matching geometry of the curved rail 378 and the channels 376A, 376B within the curved wall 374 and curved clamping body 372 results in a substantial amount of contact area between the positioning ring 338 and the ring attachment component 336, thereby ensuring a secure coupling of the ring 338 thereto such that the ring 338 is immovably coupled to the positioning device 330 when clamped to the ring attachment component 336. The incorporating of a removable positioning ring 338 and an attachment component 336 into any positioning device embodiment herein means that any ring 338 can be removed and replaced at any time for any reason. For example, in those embodiments in which a change from one ring (or ring configuration, such as any of the configurations disclosed or contemplated below with respect to FIGS. 21A-21C) to another is desirable, the attached ring can be removed and a new and/or different ring can be attached.

According one embodiment, the actuation component 390 for urging the clamping body 372 into the clamping position against the stationary wall 374 can be a lever 390 that is rotatably attached to the device base 332 at a rotational joint 392 and further is attached to the clamping body 372 via a coupling rod 394 that is disposed through an opening 396 in the stationary wall 374 and attached to the clamping body 372 as best shown in FIGS. 20D-20F. Thus, as shown in FIGS. 20B and 20E, the clamping body 372 can be positioned in the open (or “unclamped”) position when the lever 390 is positioned in its open position such that the coupling rod 394 is disposed at its furthest distance from the lever 390. Further, as shown in FIGS. 20C and 20F, the clamping body 372 can be positioned in the clamped position when the lever 390 is positioned in its clamped position such that the coupling rod 394 is disposed closest to the lever 390.

In addition, in certain embodiments, the mechanism 336 can have a tensioning component (such as a spring, for example) 396 that urges the clamping body 372 toward its open or unclamped position. As such, it can be easy to release the lever 390 and allow the body 372 to be urged into its open position, and can require additional force to urge the lever 390 (and thus the body 372) into its clamped position. Alternatively, a tensioning component can be used to urge the clamping body 372 toward its clamped position.

In various implementations, the removable positioning ring (such as ring 338 as discussed above) can have various different configurations. For example, FIGS. 21A-21C depict three different positioning device embodiments, each having a different ring configuration attached thereto. FIG. 21A shows a positioning device 400 having a positioning ring 402 that is a semicircular ring that forms a half ring (a ring structure that extends around about 180 degrees of a circle). In contrast, FIG. 21B depicts a positioning device 404 having a positioning ring 406 that is a semicircular ring that forms ¾ of a ring (a ring structure that extends around about 270 degrees of a circle). Further, FIG. 21C depicts a positioning device 408 having a positioning ring 410 that is a full circular ring that forms a complete ring.

As shown in one exemplary embodiment in FIG. 21C, a positioning ring 410 that forms a full ring allows for movement of the movable arm base 411 around the entire circumference of the ring 410, thereby providing for complete rotation of the robotic device (such as device 50) attached thereto as described elsewhere herein. This full rotation positioning can be beneficial for certain procedures that require that the arms and end effectors of the device (such as device 50) have access to all four quadrants of the patient cavity (such as the abdominal cavity, for example), such as to interact with, manipulate, and/or perform procedures on any of the tissues and/or organs in all of those areas.

Alternatively, as shown in FIG. 21A, a positioning ring 402 can be provided (for use with any positioning device embodiment herein) that forms only a partial ring—in this specific embodiment, the positioning ring 402 forms a half ring such that the movable arm base 403 is limited to about 180 degrees of movement, thereby providing for only 180 degrees of rotation of the robotic device (such as device 50) attached thereto. Such a configuration can be beneficial because, as shown in one example in FIG. 22, it allows the robotic device (such as device 50) to rotate into a position that would have been restricted by a 360 degree ring. That is, the body of the device 50 can be urged through the gap 405 in the ring 402 to achieve an angled position (in relation to the plane of the positioning ring 402) that could not be achieved if the ring were a full circle restricting the movement of the device.

Further, as shown in FIG. 21B, a positioning ring 406 can be provided (for use with any positioning device embodiment herein) that also forms only a partial ring—in this specific embodiment, the positioning ring 406 forms a ¾ ring such that the movable arm base 407 is limited to about 270 degrees of movement, thereby providing for about 270 degrees of rotation of the robotic device (such as device 50) attached thereto. As with the half ring 402 embodiment above, this configuration allows the robotic device (such as device 50) to rotate into a position that would have been restricted by a 360 degree ring. That is, the body of the device 50 can be urged through the gap 409 in the ring 402 to achieve an angled position (in relation to the plane of the positioning ring 406) in a fashion similar to that described above with respect to ring 402. In this specific exemplary implementation, the gap 409 in the ring 406 is smaller than the gap 405 discussed above such that the movable arm base 407 can rotate the robotic device more (up to about 270 degrees) in comparison to the device 400 discussed above but the ability to achieve an angled position with the robotic device (such as device 50) is comparatively more limited as a result of the comparatively smaller gap 409.

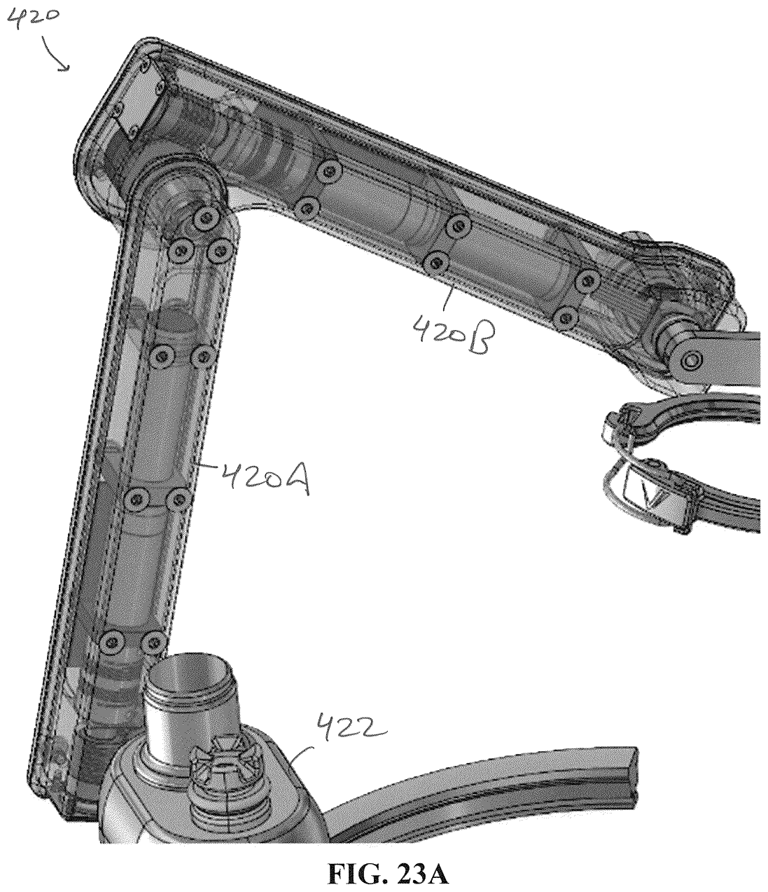

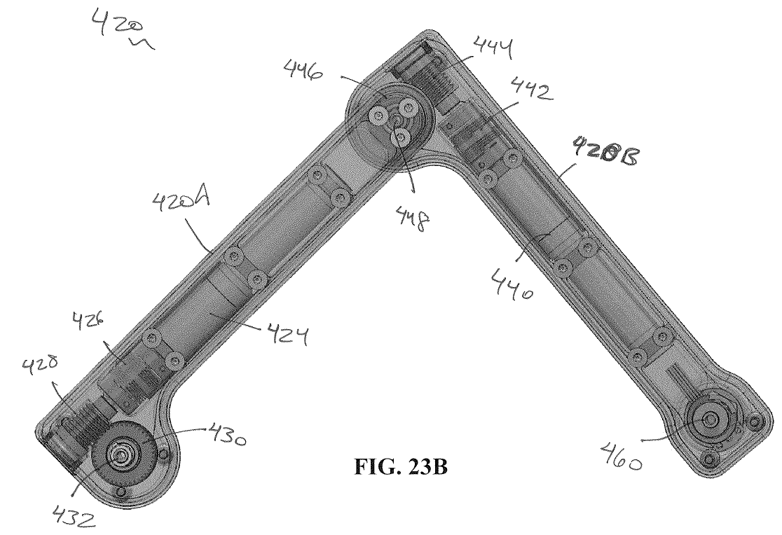

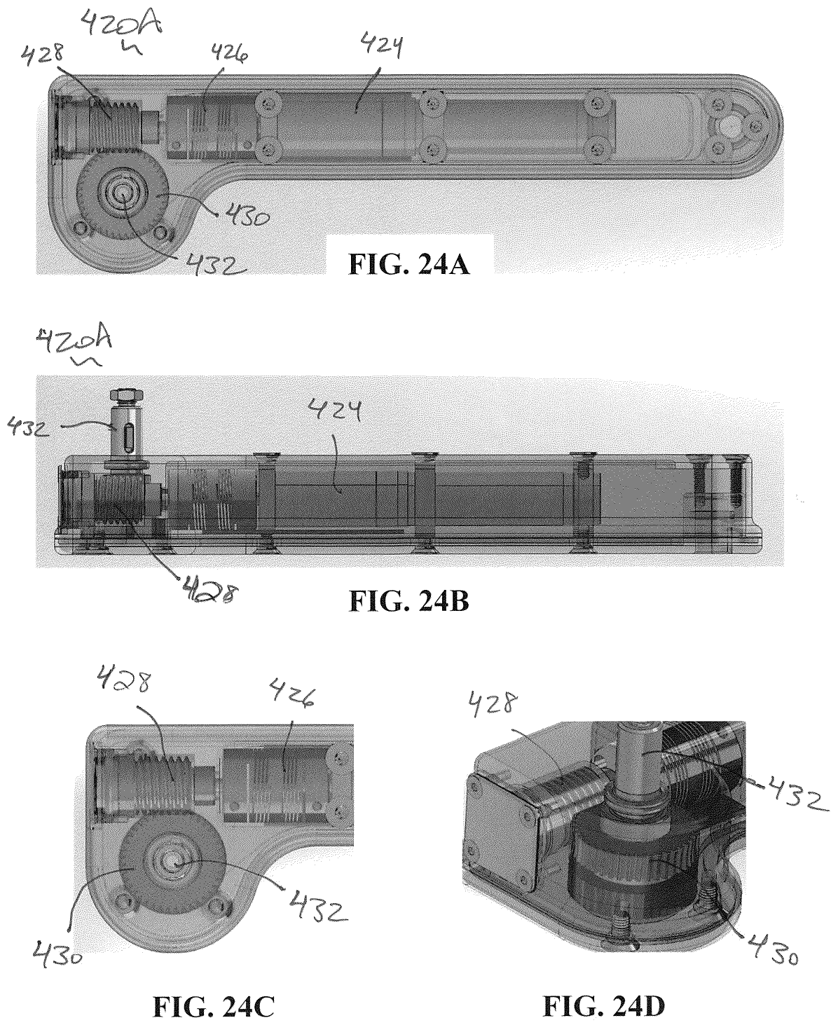

The various positioning device implementations herein have a positioning arm rotatably attached to the movable arm base, as discussed above. One specific embodiment of an arm 420 is shown in additional detail in FIGS. 23A-25. As best shown in FIGS. 23A and 23B, the arm 420 has two links 420A, 420B, with the first link 420A rotatably coupled at one end to the movable arm base 422 and the second link 420B rotatably coupled to the other end of the first link 420A.

In one embodiment, the first link 420A has a motor (or other actuator) 424 disposed within the link 420A, with a flexible shaft coupler 426 coupled to the motor 424 to account for any offset between the two coupled axels. In one embodiment, the flexible shaft coupler 426 is a slit-type flexible shaft coupling available from NBK (https://www.nbk1560.com/en-US/products/coupling/couplicon/slit_type/). Alternatively, any known flexible shaft coupler, or no such coupler, can be used. A drive gear 428 is rotationally coupled to the motor 424 such that the rotational force created by the motor 424 causes the drive gear 428 to rotate. In one embodiment, the drive gear 428 is a standard worm gear with outer threads. Alternatively, the drive gear 428 can be any known gear for transferring rotational force. The drive gear 428 is rotatably coupled to a driven gear 430. In one exemplary implementation, the driven gear 430 is a pinion gear 430. Alternatively, the driven gear 430 can be any known gear for receiving the rotational force from the drive gear 428 such that the first link 420A can rotate in relation to the arm base 422. In the embodiment as shown, the gear 430 is fixedly attached to a connecting rod 432, which is fixedly attached to the arm base 422. As such, actuation of the motor 424 causes rotation of the drive gear 428. Because the rotatable drive gear 428 is coupled to the gear 430, and because the gear 430 is fixedly attached to the arm base 422 via the connecting rod 432, the rotation of the drive gear 428 causes the arm link 420A to rotate in relation to the arm base 422 around the connecting rod 432. As such, the connecting rod 432 constitutes the joint 432 between the arm base 422 and the first link 420A around which the first link 420A rotates. Alternatively, the first link 420A can have any known configuration or assembly of at least one actuator and at least one gear to accomplish the rotation of the link 420A in relation to the arm base 422.

Similarly, the second link 420B, according to certain implementations, has a motor (or other actuator) 440 disposed within the link 420B, with a flexible shaft coupler 442 coupled to the motor 440. In one embodiment, the flexible shaft coupler 442 can be the same coupler as the coupler 426 discussed above. A drive gear 444 is rotationally coupled to the motor 440 such that the rotational force created by the motor 440 causes the drive gear 444 to rotate. In one embodiment, the drive gear 444 is a standard worm gear with outer threads. Alternatively, the drive gear 444 can be any known gear for transferring rotational force. The drive gear 444 is rotatably coupled to a driven gear 446. In one exemplary implementation, the driven gear 446 is a pinion gear 446. Alternatively, the driven gear 446 can be any known gear for receiving the rotational force from the drive gear 444 such that the second link 420B can rotate in relation to the first link 420A. In the embodiment as shown, the gear 446 is fixedly attached to a connecting rod 448, which is fixedly attached to the first link 420A. As such, actuation of the motor 440 causes rotation of the drive gear 444. Because the rotatable drive gear 444 is coupled to the gear 446, and because the gear 446 is fixedly attached to the first link 420A via the connecting rod 448, the rotation of the drive gear 444 causes the second arm link 420B to rotate in relation to the first arm link 420A around the connecting rod 448. As such, the connecting rod 448 constitutes the joint 449 between the first link 420A and the second link 420B around which the second link 420B rotates. Alternatively, the second link 420B can have any known configuration or assembly of at least one actuator and at least one gear to accomplish the rotation of the second link 420B in relation to the first link 420A.

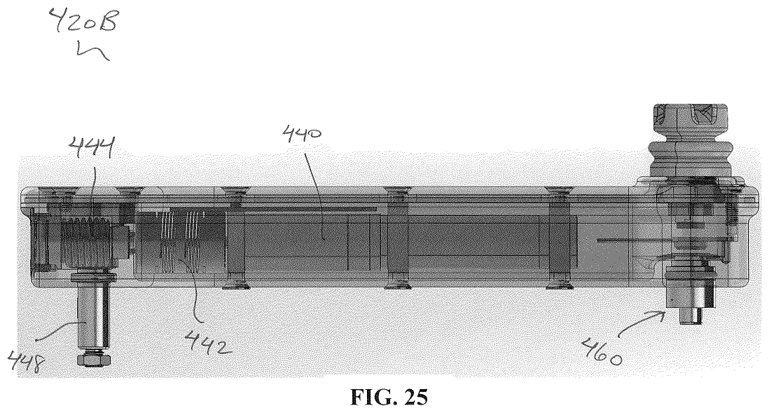

As best shown in FIGS. 23B and 25, the second link 420B also has a passive joint 460 at the end opposite the connecting rod 448. The passive joint 460 can provide a rotatable coupling to a device attachment structure (or clamp) (such as the clamp 344 discussed above). One exemplary embodiment of a passive joint (such as joint 460) and the connection to such a clamp will be discussed in further detail below.

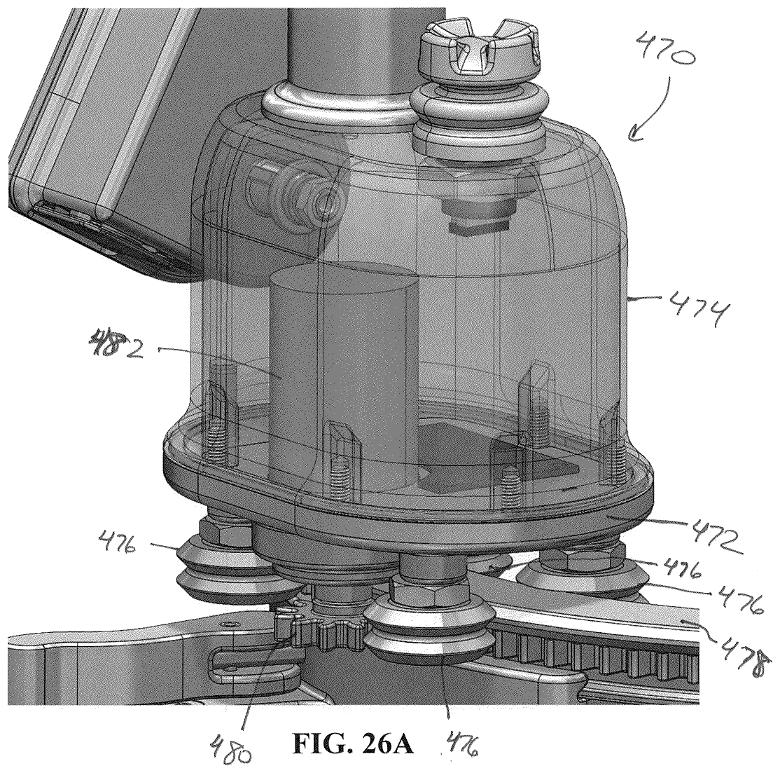

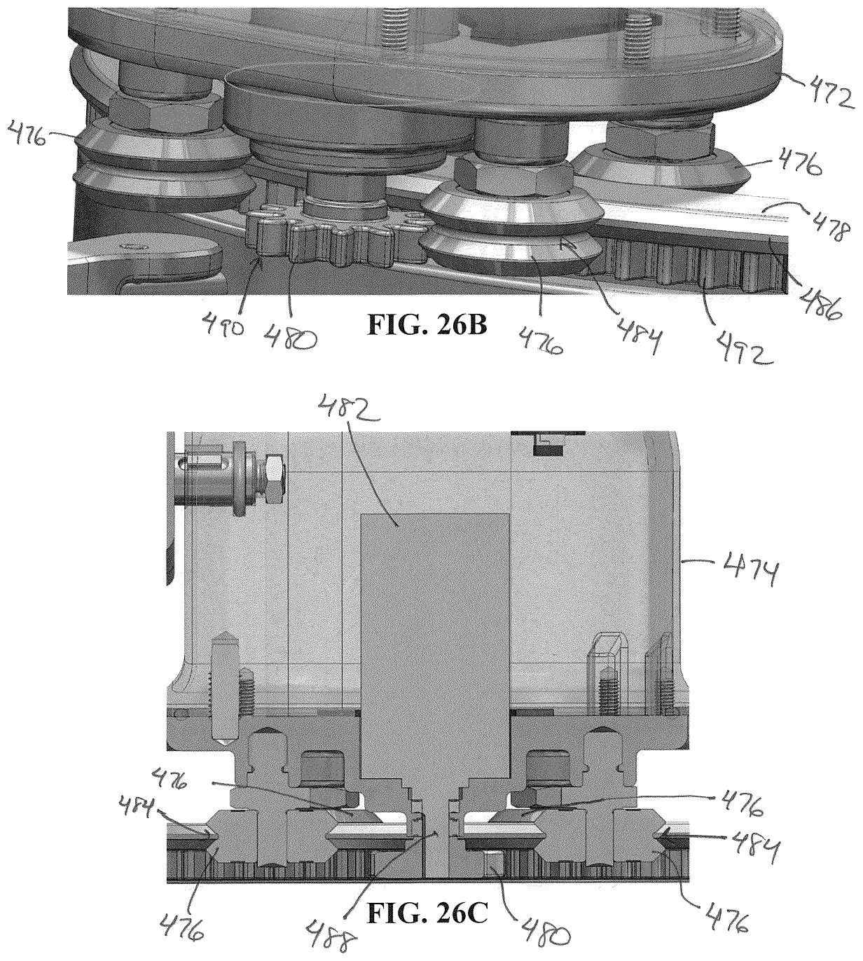

One exemplary implementation of a movable arm base 470 is depicted in FIGS. 26A-26C. In this specific version, the base 470 has a base floor 472, a housing (or enclosure) 474 that is attached to the base floor 472 and houses the internal components of the base 470, four rollers 476 rotatably attached to the underside of the base floor 472 for rollable coupling with the positioning ring 478, a drive gear 480 extending from the underside of the base floor 472 and coupled to a motor 482 (or other actuator) disposed within the housing 474. In certain embodiments, the positioning ring 478 is substantially similar to the other positioning ring embodiments disclosed or contemplated elsewhere herein.

According to one embodiment as best shown in FIGS. 26B and 26C, each of the four rollers 476 has a channel 484 defined in the outer surface of the roller 476 that can mateably couple with the protruding rib 486 extending along either side of the positioning ring 478. With two rollers 476 disposed on one side of the ring 478 and two rollers 476 disposed on the opposing side of the ring 478, the four rollers 476 securely attach the arm base 470 to the positioning ring 478 such that the base 470 can move along the length of the ring 478 as described elsewhere herein. Alternatively, any known components or mechanisms can be used to securely attach the arm base 470 to the ring 478 while allowing the base to be movable along the ring 478.

The drive gear 480, in accordance with certain implementations, is rotatably coupled to the motor 482 via a drive shaft 488 that is rotationally constrained to the motor 482 and to the drive gear 480, as best shown in FIG. 26C. As best shown in FIG. 26B, the drive gear 480 has teeth 490 that can mateably couple to the teeth 492 extending along the outer side of the positioning ring 478 (below the rib 486). As such, actuation of the motor 482 causes rotation of the drive shaft 488 and thus the drive gear 480. The coupling of the drive gear teeth 490 to the ring teeth 492 and the rotation of the drive gear 480 causes the entire arm base 470 to move along the length of the positioning ring 478, with the rollers 476 securing the base 470 to the ring 478 as it moves along the length. Alternatively, any known drive gear with any known coupling features can be used to mateably couple to corresponding coupling features on some portion of the ring 478 to allow for the base 470 to be urged along the length thereof.

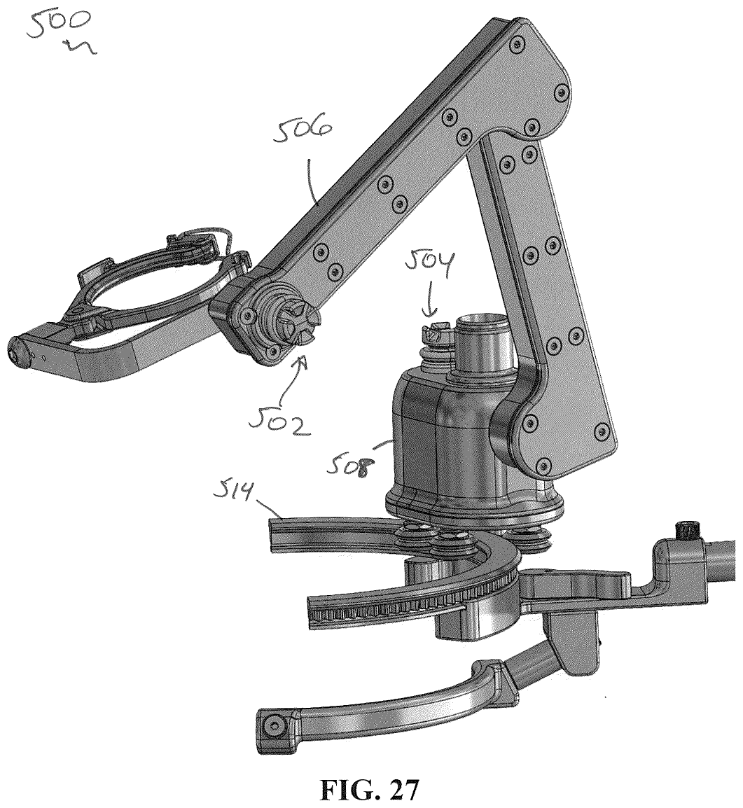

At least one alternative positioning device embodiment 500 as shown in FIGS. 27-29B can have control inputs 502, 504 disposed on the device 500 itself such that a user at the patient's bedside during a procedure can use one or both of the inputs 502, 504 to control the positioning device 500. This allows for the flexibility to control the positioning device 500 from the main console (such as the exemplary console 16 as shown in FIG. 1A), from the positioning device 500 itself, or from both. In the specific implementation as shown, the first control input 502 is disposed on the second link of the arm 506 and the second control input 504 is disposed on the arm base 508. Alternatively, the device 500 can have one or more control inputs that are disposed anywhere on the device 500.

In the version depicted, the control inputs 502, 504 are joysticks 502, 504. More specifically, both of the joysticks 502, 504 can be any of the joysticks in the TS2 Series, which is commercially available from Ruffy Controls, Inc. (https://ruffycontrols.com/ts2-series/). Alternatively, the control inputs can be handles, buttons, control knobs, or any other known control inputs for use with robotic devices.

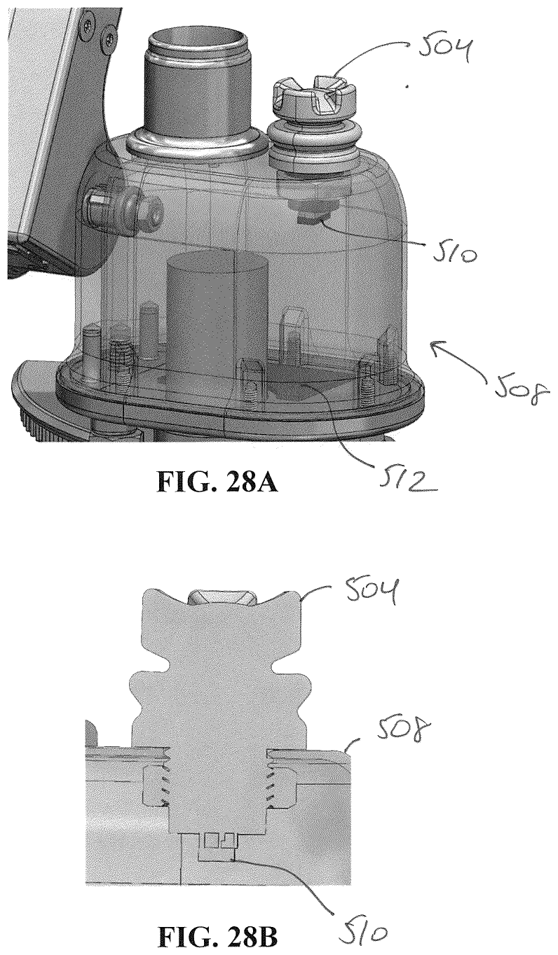

As best shown in FIGS. 28A and 28B, according to one specific embodiment, the joystick 504 is attached to the top of the arm base 508 and has a connector 510 that is electrically coupled to a processor (in this case, a circuit board) 512 via one or more wires (not shown) that extend from the connector 510 to the processor 512 such that the electrical signals from the joystick 504 are transmitted to the processor 512 via the wires. Further, the processor 512 is electrically coupled to the external console (such as console 16, for example) such that the electrical signals from the joystick 504 are transmitted to and processed by the console. Once the signals are processed at the console, the control signals are sent from the console to the appropriate actuators on the positioning device 500 (and in some cases to the actuators on the robotic device such as device 50) to cause the positioning device 500 (and in some cases the robotic device) to move as inputted at the joystick 504. In one embodiment, this joystick 504 is used to move the arm base 508 around positioning ring 514.

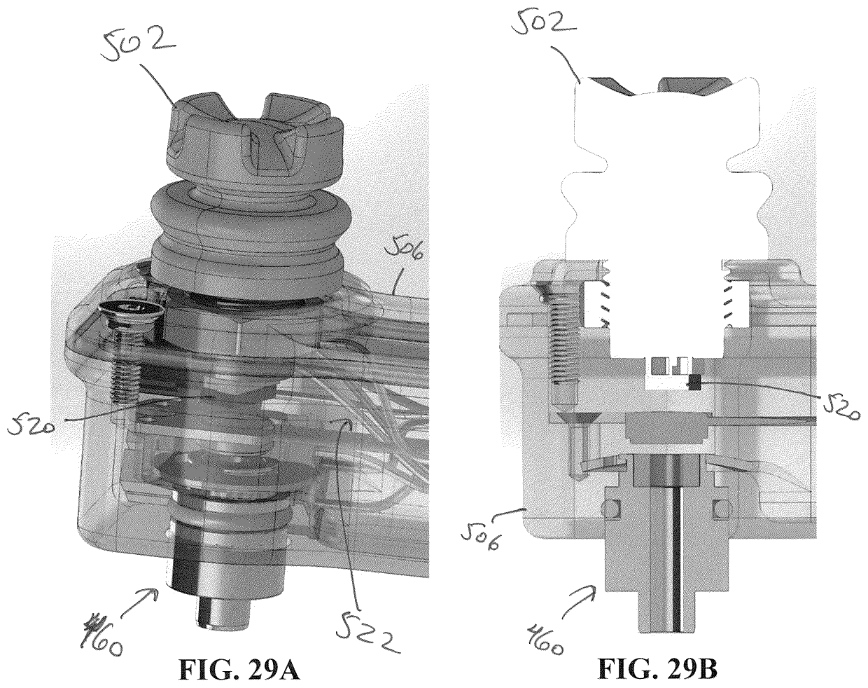

As best shown in FIGS. 29A and 29B, according to one specific embodiment, the joystick 502 is attached to the side of the second link of the arm 506 and has a circuit 520 that is electrically coupled to the external console (such as console 16, for example) via one or more wires 522 such that the electrical signals from the joystick 502 are transmitted to and processed by the console. Once the signals are processed at the console, the control signals are sent from the console to the appropriate actuators on the positioning device 500 (and in some cases to the actuators on the robotic device such as device 50) to cause the positioning device 500 (and in some cases the robotic device) to move as inputted at the joystick 502. In one implementation, this joystick 502 is used to move the arm 506.

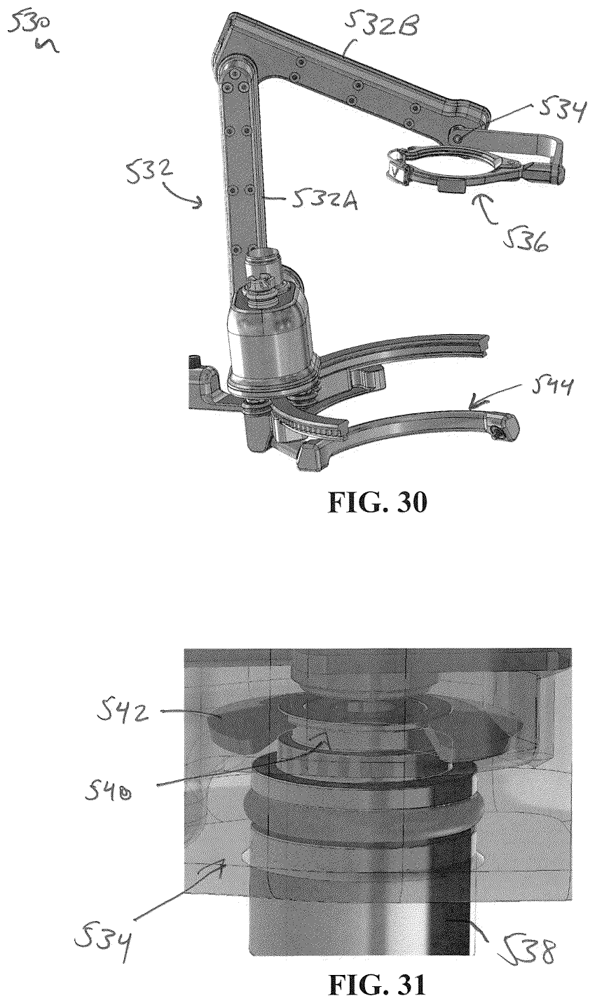

As mentioned above, certain positioning device embodiments such as the positioning device 530 as shown in FIG. 30 can have a passive joint 534 that rotatably couples a device attachment structure (or clamp) 536 to the second link 532B of the positioning arm 532. In some implementations, the passive joint 534 is substantially similar to the passive joint 460 discussed above. As best shown in FIG. 31, in one embodiment, the passive joint 534 is a passive friction joint 534 that is made up of a rotatable connecting rod 538 that is rotatably coupled to the second link 532B. The rod 538 has a groove 540 defined therein at or near the proximal end of the rod 538 such that a tensioned friction ring 542 can be positioned within the groove 540. In one specific implementation, the tensioned friction ring 542 is a “bowed side-mount external retaining ring” 542, which is commercially available from McMaster-Carr (https://www.mcmaster.com/98398A134/). Alternatively, any friction ring or friction component can be used. The tensioned friction ring 542 creates friction between the rod 538 and the link 542B (or an internal portion thereof), thereby resisting easy rotation of the clamp 536 in relation to the second link 532B. According to certain embodiments, the passive friction joint 534 makes it possible for the clamp 536 to rotate with the body of the device (such as device 50) to which it is clamped as the device is disposed through the port (not shown) coupled to the port support structure 544 and positioned by the positioning device 530.

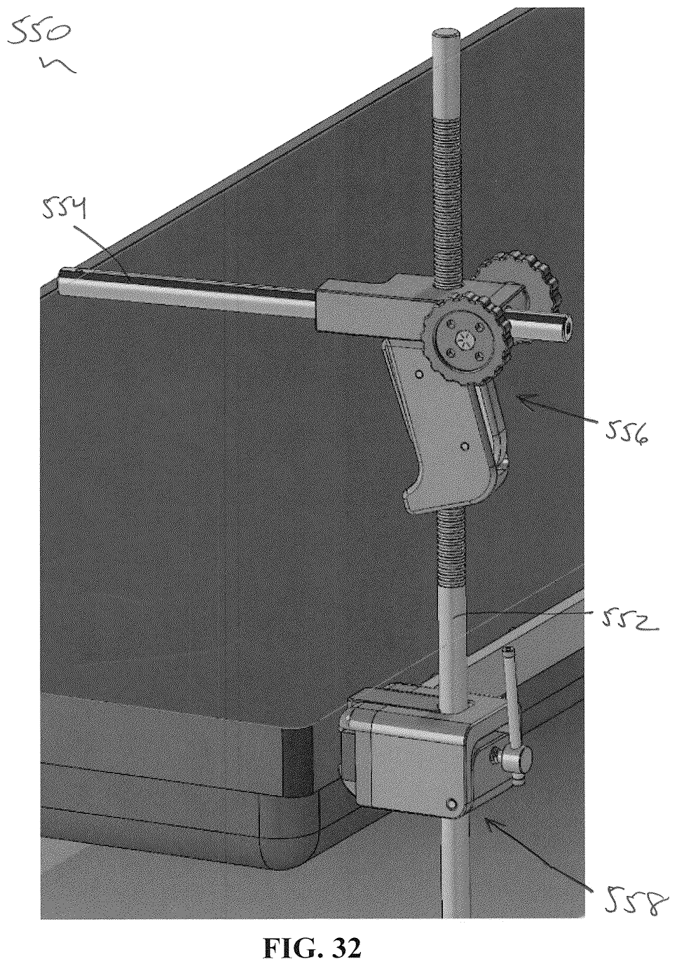

Another support arm embodiment 550 is depicted in FIGS. 32-34E that can be used to attach the any positioning device embodiment disclosed or contemplated herein to the surgical bed and allow for pre-operative positioning of the system. The arm 550 has an elongate vertical (or “first”) rod 552 that is coupled at one end (its “bottom” end) to the standard rail of the surgical table (or any known part of the table). At the second (or “top”) end, the rod 552 is adjustably coupled to a horizontal (or “second”) rod 554 via an adjustment device 556 that can adjustably control the positioning of the two rods 552, 554 in relation to each other as explained in detail below. Further, the support arm can also have a rail attachment device 558 that can be used to attach the bottom end of the vertical rod 552 to a standard railing of the surgical table. It is understood that the first and second rods 552, 554 can be any elongate structures, including tubes, pipes, bars, or any other such structures. In this exemplary embodiment, the support arm 550 allows for four degrees of freedom relative to the surgical table: vertical translation, horizontal translation, rotation about the vertical rod 552, and rotation about the horizontal rod 554.

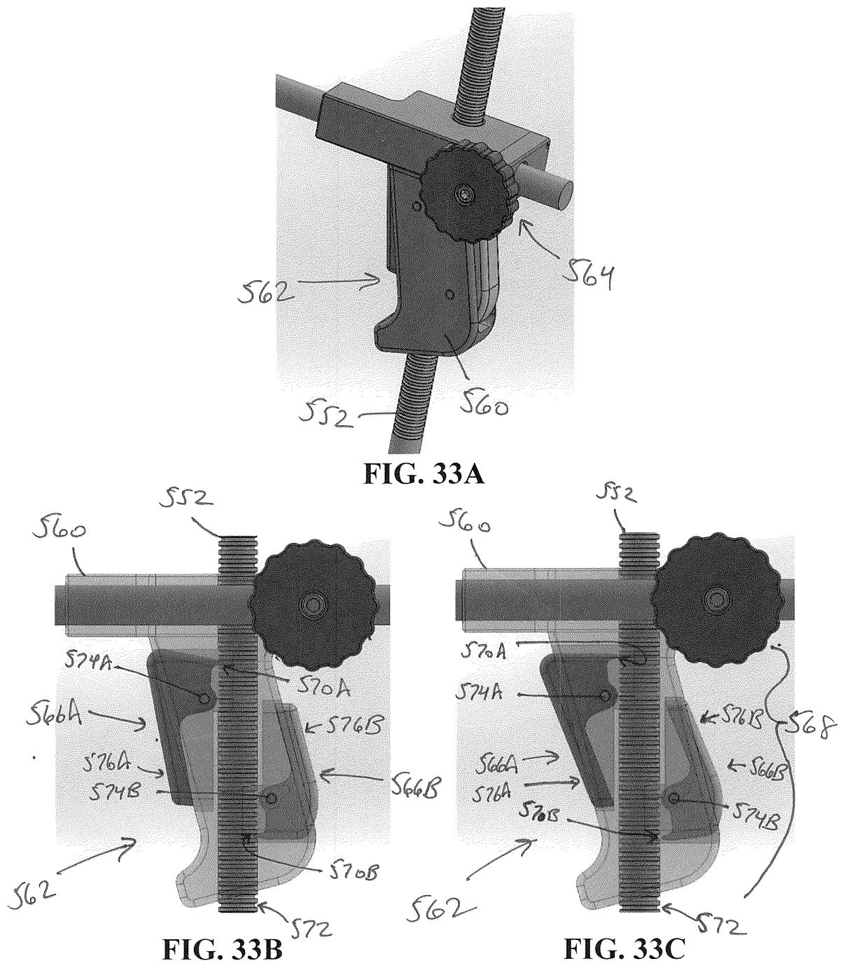

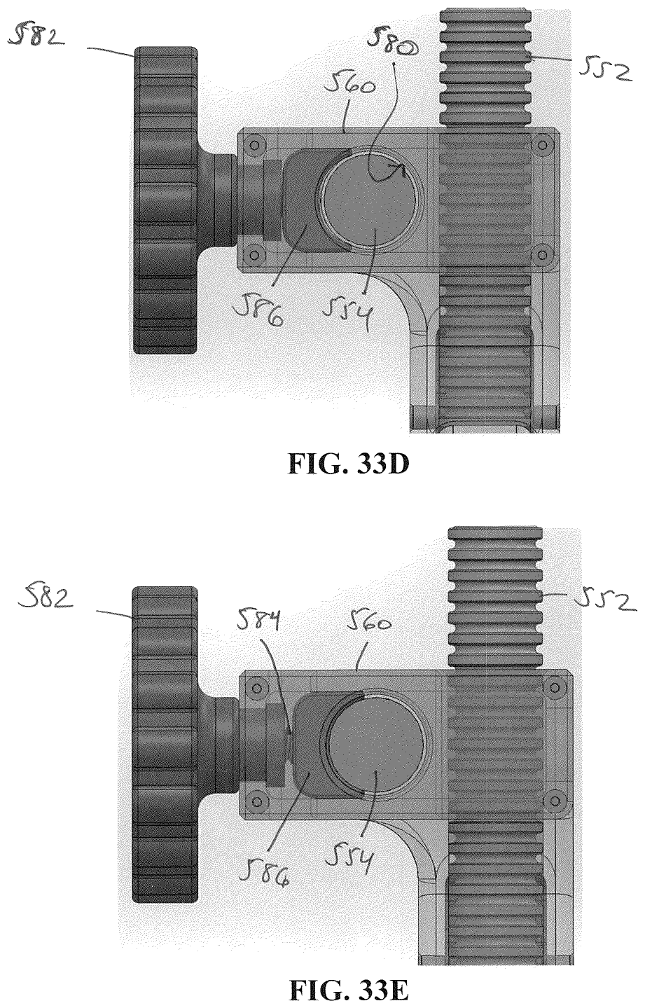

One specific embodiment of an adjustment device 556 is depicted in FIGS. 33A-33E. The device 556 has a housing 560 that contains both a height adjustment mechanism 562 (along the vertical rod 552) and a horizontal bar adjustment mechanism 564 (along the horizontal rod 554).

The height adjustment mechanism 562, according to certain implementations, is depicted in further detail in FIGS. 33B and 33C. The mechanism 562 consists of two actuation structures (also referred to herein as “triggers”) 566A, 566B disposed on opposing sides of the handle portion 568 of the housing 560, along with a lumen (not shown) defined through the housing 560 such that the vertical rod 552 is disposed between the two triggers 566A, 566B. Each of the triggers 566A, 566B has protrusions 570A, 570B extending from the triggers 566A, 566B that can be positioned in slots 572 defined in the outer surface of the vertical rod 560 such that the triggers 566A, 566B and thus the housing 560 are attached to the rod 560 at a desired location along the length of the rod 560. Each of the triggers 566A, 566B also has a rotational joint 574A, 574B rotatably attached to the housing 560 and the trigger 566A, 566B that the trigger 566A, 566B rotates around in relation to the housing 560. As such, each trigger 566A, 566B can be actuated or depressed by a user at an actuable section 576A, 576B to rotate the trigger 566A, 566B around its rotational joint 574A, 574B, thereby causing the protrusion 570A, 570B to move in relation to the rod 552. In certain embodiments, each trigger 566A, 566B has a tensioning component (not shown) that applies tension urging the trigger 566A, 566B into the locked position in which the protrusion 570A, 570B is disposed within a slot 572 in the rod 552, thereby locking the adjustment device 556 onto the rod 552 at a desired position along the length thereof. That is, each trigger 566A, 566B is disposed in its untensioned or resting state in the locked position.

As such, in order to move the adjustment device 556 to a different position along the length of the vertical rod 552, a user must grasp the handle portion 568 of the device 556 and depress the actuable section 576A, 576B of each trigger 566A, 566B with sufficient force to overcome the tensioning component (not shown) and cause the trigger 566A, 566B to rotate such that the protrusion 570A, 570B of each trigger 566A, 566B is urged out of the slot 572. At this point, the device 556 can be moved vertically along the length of the rod 552 to a desired position. Once the device 556 is positioned as desired, the user can release the actuable portions 576A, 576B and the tensioning component will urge the protrusions 570A, 570B into the closest slots 572, thereby locking the device 556 into place on the rod 552.

The use of two independent triggers 566A, 566B can be used as a safety feature. That is, because the two triggers 566A, 566B are independent from each other, both must be simultaneously actuated such that both protrusions 570A, 570B are urged out of their slots to allow for vertical movement of the device 556. As such, the two independent triggers 566A, 566B limit the possibility of unexpected movement of the device 556 as a result of accidentally pressing/bumping one of the triggers 566A, 566B.

The horizontal bar adjustment mechanism 564, according to certain implementations, is depicted in further detail in FIGS. 33D and 33E. The mechanism 564 consists of a lumen 580 defined through the housing 560 such that the horizontal rod 554 is disposed therethrough. Further, the mechanism 564 includes a rotatable component (or “knob”) 582 that is rotatably coupled to the housing 560 such that its rotational axis is transverse to the axis of the lumen 580 (and thus the horizontal rod 554). In addition, the knob 582 has a threaded rod 584 extending therefrom that is threadably coupled to a frictional fixation body 586 that is in communication with the lumen 580 and is slidable along the axis of the knob 582. As such, the knob 582 can be used to adjust the position of the device 556 along the length of the horizontal bar 554. That is, to release the device 556 from the bar 554, the knob 582 can be rotated in one direction such that the frictional fixation body 586 is urged away from the bar 554, thereby allowing the bar 554 to slide freely within the lumen 580. Further, to attach the device to the bar 554 at a desired location, the knob 582 can be rotated in the opposite direction such that the frictional fixation body 586 is urged toward and into frictional contact with the bar 554 until the frictional contact is sufficient to fixedly attach the device 556 to the bar 554.

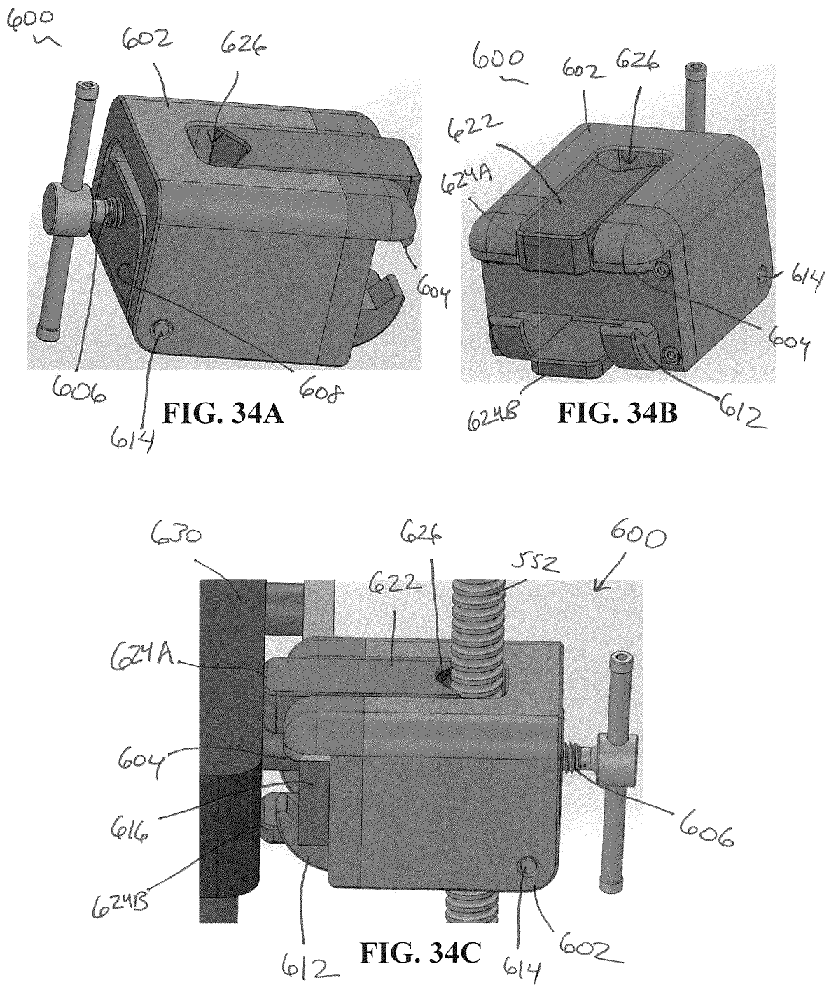

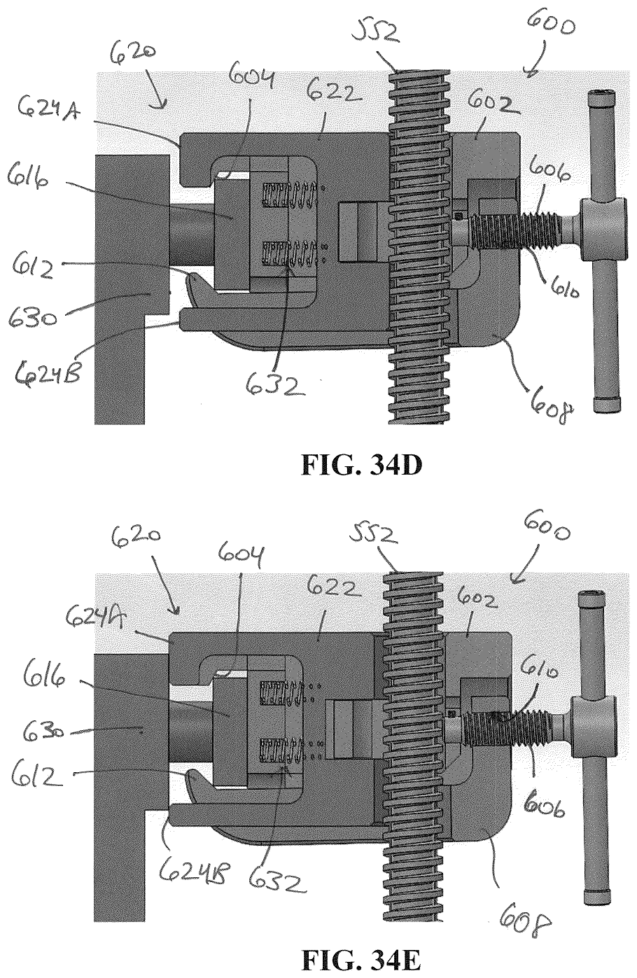

One exemplary embodiment of a rail attachment device 600 that can be used to attach the bottom end of the vertical rod 552 to a standard railing of the surgical table is shown in detail in FIGS. 34A-34E. The attachment device 600 has a device body 602 with upper rail attachment hooks 604 extending from the distal end of the body 602 and a clamping screw 606 that is rotatably coupled to a proximal end of the body 602. The upper rail attachment hooks 604 can be positioned in contact with the upper edge of the table rail 616 such that the hooks 604 are engaged therewith.

In addition, the device 600 has a movable clamp body 608 that is disposed within the sides of the device body 602 (as best shown in FIG. 34B), with a threaded lumen 610 at its proximal end that receives and threadably couples to the clamping screw 606 (as best shown in FIGS. 34D-E) and lower rail attachment hooks 612 extending from the distal end of the movable clamp body 608 such that the hooks 612 extend beyond the distal end of the device body 602 and are disposed below the upper rail attachment hooks 604. The movable clamp body 608 is rotatably coupled to the sides of the device body 602 at a rotational joint 614 as best shown in FIGS. 34A-34C. As such, the clamp body 608 can rotate via the rotational joint 614 by rotating the clamping screw 606 such that the lower rail attachment hooks 612 move between a lower or unclamped position and a raised or clamped position in which the hooks 612 are in contact with the lower edge of the table rail 616.

Thus, the device body 602 can first be attached to the top edge of the rail 616 by urging the distal end of the device body 602 into contact with the rail 616 such that the upper rail attachment hooks 604 are engaged with the top edge of the rail 616 (as shown in FIGS. 34C-E). At this point, the lower rail attachment hooks 612 are disposed in the unclamped position. Once the device body 602 is engaged with the top edge of the rail 616, the clamping screw 606 can be rotated to urge the proximal end of the movable clamp body 608 proximally, thereby causing the body 608 to rotate around the joint 614 such that the lower rail attachment hooks 612 at the distal end of the clamp body 608 are raised or otherwise urged upward (toward the rail 616) until the lower rail attachment hooks 612 make contact with and engage with the lower edge of the rail 616, thereby clamping the rail attachment device 600 to the rail 616.

In addition, the rail attachment device 600 also has a table engagement component 620 that is made up of an engagement component body 622 and the table engagement protrusions 624A, 624B at the distal end of the component 620 that can be urged into contact with the side of the surgical table 630. The table engagement component 620 is slidably disposed within the device body 602 such that it can move between a retracted position (as shown in FIG. 34D) and an extended or contact position (as shown in FIG. 34E) in which the protrusions 624A, 624B are in contact with the side of the table 630. The device 600 has one or more tensioning components (such as, for example, springs) 632 (as best shown in FIGS. 34D-E) that are coupled to the table engagement component 620 and the device body 602 such that they urge the component 620 into its retracted position. Further, the proximal end of the table engagement component 620 in combination with a slot in the device body 602 forms a lumen 626 through which the vertical rod 552 can be disposed. As such, as the clamping screw 606 is tightened, the distal end of the screw 606 pushes the rod 552 distally toward the table and into the engagement component body 622, thereby urging the body 622 toward and into contact with the table 630 as shown in FIG. 34E. Thus, tightening the screw 606 not only causes the clamp 600 to be tightly clamped to the table rail 616 as described above, but also urges the table engagement component body 622 into the table 630 and thereby provides a more stable attachment to the rail 616 and the table 630 in comparison to known clamps that only attach to the rail.

To reiterate, the ability of the table engagement component 620 to contact the side of the surgical table 630 results in the clamping device 600 engaging with both the rail 616 and the side of the table 630. As a result, the clamping device 600 achieves a more stable attachment to the surgical table 630 in comparison to known clamping devices that engage solely with the rail. This is in part because the rigidity of the rail 616 alone can be less than that of the surgical table 630 itself. In addition, given the high loads and precision positioning that can be required for the systems attached to the support arm 550, a stable and strong connection to the surgical table 630 can reduce unintended movement of whatever system is attached to the support arm 550.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

Although the various embodiments have been described with reference to preferred implementations, persons skilled in the art will recognize that changes may be made in form and detail without departing from the spirit and scope thereof.

What is claimed is:

1. A gross positioning system for use with a robotic surgical device, the system comprising:
(a) a positioning ring;
(b) an arm base rotatably coupled to the positioning ring;
(c) an arm assembly operably coupled to the arm base, the arm assembly comprising a first link rotatably coupled to the arm base and a second link rotatably coupled to the first link; and
(d) a device clamp rotatably coupled to the second link, wherein the device clamp is sized to receive a body of the robotic surgical device therein such that the body is releasably clamped thereto.

2. The gross positioning system of claim 1, wherein the arm base comprises a base actuator rotatably coupled to the positioning ring.

3. The gross positioning system of claim 1, wherein the first link comprises a first arm actuator disposed within the first link and rotatably coupled to the arm base and the second link comprises a second arm actuator disposed within the second link and rotatably coupled to the first link.

4. The gross positioning system of claim 1, further comprising a port support arm disposed adjacent to the positioning ring.

5. The gross positioning system of claim 4, further comprising a port attachment structure rotatably attached to the port support arm, wherein the port attachment structure is removably coupleable to a port.

6. The gross positioning system of claim 4, wherein the port support arm is coupled to the gross positioning system via a connection rod, wherein the connection rod is disposed at an angle in relation to a plane of the positioning ring, wherein the angle can range from about 0 degrees to about 45 degrees.

7. The gross positioning system of claim 5, wherein the port attachment structure is rotatable in relation to the port support arm at an angle in relation to the port support arm that ranges from about 0 degrees to about 45 degrees.

8. The gross positioning system of claim 4, wherein the positioning ring and the port support arm are sized to receive a portion of the robotic surgical device movably positioned therethrough.

9. The gross positioning system of claim 1, wherein the positioning ring comprises a semicircular ring.

10. A gross positioning system for use with a robotic surgical device, the system comprising:
(a) a stationary positioning ring;
(b) an arm base moveably coupled to the positioning ring, wherein the arm base comprises a base actuator rotatably coupled to the positioning ring;
(c) an arm assembly operably coupled to the arm base, the arm assembly comprising:
(i) a first link rotatably coupled to the arm base, the first link comprising a first arm actuator disposed within the first link and rotatably coupled to the arm base; and
(ii) a second link rotatably coupled to the first link, the second link comprising a second arm actuator disposed within the second link and rotatably coupled to the first link;
(d) a device clamp rotatably coupled to the second link, wherein the device clamp is sized to receive a body of the robotic surgical device therein such that the body is releasably clamped thereto; and
(e) a port support arm disposed distal and adjacent to the positioning ring, the port support arm comprising a port attachment structure rotatably attached to the port support arm, wherein the port attachment structure is removably coupleable to a port.

11. The gross positioning system of claim 10, wherein the port support arm is coupled to the gross positioning system via a connection rod, wherein the connection rod is disposed at an angle in relation to a plane of the positioning ring, wherein the angle can range from about 0 degrees to about 45 degrees.

12. The gross positioning system of claim 10, further comprising a port attachment structure rotatably attached to the port support arm, wherein the port attachment structure is removably coupleable to a port.

13. The gross positioning system of claim 12, wherein the port attachment structure is rotatable in relation to the port support arm around an axis disposed at an angle in relation to the port support arm that ranges from about 0 degrees to about 45 degrees.

14. The gross positioning system of claim 10, wherein the positioning ring and the port support arm are sized to receive a portion of the robotic surgical device movably positioned therethrough.

15. The gross positioning system of claim 10, wherein the positioning ring comprises a semicircular ring.

16. The gross positioning system of claim 15, wherein the semicircular ring comprises a ring structure that extends around about 180 degrees of a circle or a ring structure that extends around about 270 degrees of a circle.

17. The gross positioning system of claim 10, wherein the positioning ring comprises a full ring.

18. A support arm, the arm comprising:
(a) a vertical rod;
(b) a horizontal rod;
(c) an adjustment device coupling the horizontal rod to the vertical rod, the adjustment device comprising:
(i) an adjustment device body;
(ii) a vertical rod lumen defined through the adjustment device body, wherein the vertical rod is moveably disposed within the vertical rod lumen;
(iii) first and second triggers operably coupled to the adjustment device body on opposing sides of the vertical rod lumen, wherein each of the first and second triggers is operably coupleable with the vertical rod;
(iv) a horizontal rod lumen defined through the adjustment device body, wherein the horizontal rod is moveably disposed within the horizontal rod lumen; and
(v) a rotatable component rotatably coupled to the adjustment device body, wherein a portion of the rotatable component is rotatably positionable in the horizontal rod lumen such that the portion is engageable with the horizontal rod;
(d) a rail attachment device operably coupled to the vertical rod, the rail attachment device comprising:
(i) a rail attachment device body comprising upper rail attachment prongs extending from a distal end of the rail attachment body;
(ii) a moveable clamp body movably attached to the rail attachment device body, the moveable clamp body comprising lower rail attachment prongs extending from a distal end of the moveable clamp body;
(iii) a clamping screw threadably coupled to the rail attachment device body and the moveable clamp body;
(iv) a table engagement body slidably coupled to the rail attachment device body; and
(v) a vertical rod lumen defined by the rail attachment device body and the table engagement body, wherein the vertical rod is moveably disposed within the vertical rod lumen,
wherein rotation of the clamping screw in one direction causes the moveable clamp body to move into a clamped position and urges the vertical rod into the table engagement body such that the table engagement body is moved into a table engagement position.

19. The support arm of claim 18, wherein the moveable clamp body is rotatably attached to the rail attachment device body, wherein the moveable clamp body is rotatable between an unclamped position and the clamped position.

20. The support arm of claim 18, wherein the moveable clamp body is in the clamped position, the upper rail attachment prongs are configured to be disposed in contact with an upper edge of a surgical table rail and the lower rail attachment prongs are configured to be disposed in contact with a lower edge of the surgical table rail.
